SEQUENCE LISTING (10 sequences)

SEQ ID NO: 1 - DNA, 7606 bp, Homo sapiens
aagaatcagaagacatttgtgctttggggagcagaggccctcagggtatagagaaggaagaagagagaggttcactgtagtcctgaagcagaaataagacctgtggctgaaggaagccttagcaattcactccttcctcttcctgagaactctctgtaggaagtctcacctagcagaggcttcacagtatttcagagaagccaaagattgtttgcctctttggaaactgttatccttccatcatgactgtgtcactcctgccactgttccaccatagagatggcgtcctttgcagcaaaccgtaagttataaggatgagggaagaagagtagagggccaaaaggattccattttgaggaaaaactacagtttgccttgccaggtagaagaatcaggcgcccagacaccatgtcacaaccctccagaactgacgttggcaggaagtagagactttgttgcctgtgtcccccatcctcaccatgtcttcgactcagggcaatggggaacactggaagtccctggagtcagtgggcatcagccgcaaagagctagcgatggccgaagccctgcagatggagtatgatgccctgtcccggctccggcatgacaaggaggagaacagagccaagcagaacgcagacccctctctcatcagctgggatgagcctggggtagacttttacagcaagccagcaggaaggcggaccgacctcaagctgttacgcggtctctctggctctgatcctacccttaactacaactcactctccccacaggaagggccgcccaaccactctacctcccaagggccacagcctggctcagatccctggcccaaaggctccctgtctggagactatctctacatttttgatggttcagatgggggagtctcttcgtccccaggaccaggggacatagagggctcttgcaagaaactatccccacctcctctgcctccccgagcttctatctgggatacccctcccctgcctcccagaaaggggtccccctcatcctccaagatctcccagcccagtgacatcaacactttctctttggtcgaacaattgccgggcaaactgctagagcatcggatcctagaagaggaagaggtgctgggaggtgggggtcaggggcgcctactggggtctgtggactatgatggtatcaatgatgcaattactaggctcaacttgaaatcgacctatgatgcggagatgttgcgggatgccaccaggggctggaaggagggccgagggccgctggacttcagcaaagacacctctggaaaacccgtggccaggagcaagactatgccccctcaggtgcccccccgcacctatgcctcccgctatggcaaccgaaagaatgcgacgcctggcaagaaccgccggatttctgcagccccggtgggctcccggccccacactgttgccaatggccatgagttgtttgaggtctcagaagagagagatgaggaggttgctgcattttgccacatgctggatatccttcgatctggctctgacatccaagactacttcctcactggctatgtctggagtgctgtcacccctagcccagagcacctcggggatgaggtcaacctgaaggtgactgtgttgtgtgacaggcttcaagaggcactcactttcacctgcaactgttcctccactgtagacttgcttatctaccagaccctgtgctacacccatgatgacctgaggaatgtggacgtgggtgactttgtgctaaagccctgcgggctggaggagttcctgcagaacaagcatgccttgggcagtcatgagtacatccaatactgccgcaagtttgacattgacattcggctacagctgatggagcagaaggttgtgcgcagtgacctggcccggacggtgaatgatgaccagagcccctccaccttgaactacctcgtccatctccaagagaggcctgtcaagcagaccatcagcaggcaggccctgagtcttctgttcgacacttaccacaatgaggtggatgccttcctgctggctgatggagacttcccactgaaggctgacagggtggtccagtccgtcaaggccatctgcaacgccctggccgccgtggaaacccctgagatcaccagtgctctcaaccagctgcccccctgcccctcccgcatgcagcctaaaattcagaaggatcccagtgtcttggctgtgagggaaaaccgagagaaggtcgtggaagccctgaccgctgccatcttggacctggtggagctgtactgcaacacattcaacgcagacttccagacggcagtgcccgggagccgcaagcatgacctggtccaggaggcctgccatttcgccaggtccctggccttcactgtctatgccacccaccgcatccccatcatctgggctaccagctatgaagatttctacctctcctgctccctcagccatggcggcaaggagctgtgcagccccctgcagacccgaagagctcacttctccaagtacctcttccacctcatcgtctgggaccagcagatctgcttcccagtgcaggtgaaccggctgcctcgggagacactgctgtgtgccactctctatgctctgcccatccccccaccggggagctcctcagaggccaataagcagcggcgggtgcctgaagccctgggctgggtcactaccccactcttcaacttcaggcaggtcctgacctgtggccggaagcttctgggtttgtggccagcaacacaggaaaatcccagcgcccgttggagtgcacctaatttccaccagccagacagtgtcatcctgcagattgacttccccacctcggcctttgacatcaagttcaccagcccccctggagacaagttcagcccccgctatgagtttggcagcctccgggaagaagaccagcgcaagcttaaagacatcatgcagaaagagtccttgtactggctcactgatgctgacaagaagcgcctgtgggagaagcgatattactgccactcggaggtgagctcgctccccctggtgctcgccagcgcccccagctgggagtgggcttgcctgcctgacatctatgttctcctgaagcagtggacccacatgaaccaccaggatgccctggggctcctgcatgccaccttcccggaccaggaggtgcgtcgtatggctgtgcagtggattggctcactctcagatgctgagctgctagactacctgccccagctggtacaggccctgaagtatgaatgctacctggacagcccgttggtgcgcttcctcctgaaacgagctgtgtctgacttgagagtgactcactacttcttctggttactgaaggacggcctcaaggactctcagttcagcatccgctaccagtatctgctggcagccttactgtgctgctgtggcaaggggctgagagaagagtttaaccgccagtgctggcttgtcaatgccctggccaaactggcccagcaggtccgggaggcagccccatctgcaaggcagggaatcctccgcacgggcctggaggaggtgaagcagttctttgccctcaatggctcgtgccgcttgccactcagccccagtctgctggttaagggaattgtgcccagggactgttcctacttcaactccaatgctgtccccctcaaactctccttccaaaatgtggatcccctgggtgagaacatccgtgtcatcttcaagtgtggggacgaccttcgccaggacatgctaacgctgcagatgattcgcatcatgagcaagatctgggtccaggaggggctggacatgcgcatggtcatcttccgctgcttctccaccggccggggcagagggatggtggagatgatccctaatgctgagaccctgcgtaagatccaggtggagcatggggtgaccggctcgttcaaggaccggcccctggcagactggctgcagaaacacaaccctggggaggacgagtatgagaaggctgtggagaactttatctactcctgcgctggctgctgcgtggccacgtacgtcttgggcatctgtgaccgacataatgacaacatcatgctgaagaccactggtcacatgttccacattgattttggccgcttcctgggccatgcccagatgtttggcaacatcaagcgggaccgtgccccctttgtcttcacctcggacatggcgtatgtcatcaacgggggtgacaagccttccagccgcttccatgattttgttgacctttgctgccaagcctacaacctcattcgcaagcacacccacctcttcctcaaccttctgggcctgatgttgtcctgtgggatccctgaactctcagacctggaggacctcaagtatgtgtacgatgccctgaggcctcaggatacagaggccaatgccactacctacttcactaggttgattgagtccagcctgggcagtgtagccacaaagctcaattttttcatccataatctggctcagatgaagttcacgggctcagatgaccggctgaccctctcctttgcctcccgaacacacactctcaagagctctggccgaatcagtgatgttttcctctgccgccatgagaagatcttccaccccaacaaaggctatatatatgtggtaaaggtgatgcgagagaacactcacgaggccacctacatccagcggacctttgaggagttccaggaattacacaataagttgcggctgctcttcccttcttcccacttgcccagcttccctagtcgcttcgtgatcggccgctcccggggagaggcggtggccgagcggcggagggaggagctaaacggttacatctggcacttgatccacgcaccccctgaggtggccgagtgtgatttggtgtacaccttcttccacccactgccccgggatgagaaggctatgggcaccagcccagctcctaagtcctcagatggcacatgggcccggcccgtcggaaaggtgggaggggaggtgaagctgtccatctcctacaaaaacaataaactcttcatcatggtgatgcatattcggggcttgcaactgctccaggatggaaatgaccctgacccctatgtgaaaatttacctccttcctgaccctcagaaaaccactaagaggaaaaccaaagtggcccggaaaacctgcaatcctacctacaatgagatgttggtatatgatgggatccccaagggtgacctgcagcagcgggagctccagctgagcgtgctgagtgagcagggattctgggagaacgtcctcctcggtgaggtgaacatccgcctgcgagagctggacctggctcaggagaagaccggctggttcgccctgggatctcgaagtcatggcaccttgtgagcccagcagagccaccacccagcatcccaggctggtggcaggagctgggggagaggactctcccctgtgagactcctccttgtgaagggccagggccctgggcaggcctccagctcggtccaggtgattctggcctctgtggtaggaggcagggagagtaagacatgctctgctgtctcttcctctggagactgaacttgggttggttgtgatgagcagccccttggaggctgtgaggttgcagcaaagttttaagtttaccttgtgtcaagggagcaatgcttggtttggggagtgtgtggggtgggctgtatgaagtagcattttgggggtgggtgggtggatatcttaatttttatttttaaaaaatgaaatagtgatgttgtcctaactgggacaggaagccttgcgagaagggacgtacctatgccccagaaggcaagagaggaagactatttggactttttgtatgattaaggttcttattggacttttccctaggttttttttttttgttattgttgttgttgtttcgttttctagctataggaactatctggggaggggcccagtgggtcctcggccagagccctctctaaggacaggttggggagggttggggagggctgcctgtgctggactgaggcttgtgccactgggcctttctgattttgcctccaaaggagagcgctgtgatacctacatgtgtaaggaagggccttccgtattggggttctgccaaggacccgtattcagggacccatgctcttttggggggacttttcctcttgtcttccctactttattaggacttgccctgaataccattttctaccccttgcccctccattctcctggcccttctgggggtcagctggtctctatgaatatgctgggggtgcttccccataggtctctcccttcatttgtctctggtgggacaaaatactgactcagtccttagatgtagtttcacccaagagcatcttggccctgggaagaggtccctaggctgcagatgctactgactgcttgctaggtagcctctggaaagcattccccatccatcactccccacttctttctgctgtgctgcttccctcccaaactccatttctgtcaccctttttataagacttttcctcattctgtggggccataaacctatttagtctggagccaaagggatgccctatctgaaggaaaggggcatggggtgggggattccatcaaaactgttgttttttgccccatgatttttctttggtcagtaggaggctggattggagtggtgattattcccctggagctaagctcaggagcccgaagggagagactgagactgactcccttatctcttcatattctttattccctaccagatggatttttttttttttttttggagacggagtctcgccctgtcgccaggctggagtgtagtggcatgatctcgactcactgcaaaatctgcctcccgggttcaagcgattctcctacctcagcctcccgagtagctgggattacaggcatgtgccaccacgccaagctaatttttgtatttttagtagagacggggtttcaccatgttggccaggatggtctcgatctcttgacctcgtgatctgcctgccttggcctcccaaagtgctgggattacaggcgtgagccaccatgccccgccccagatggattttacatttgctcttttgtgtttcgctccaaagggttgtcttcctcgccaaaaggagggagggactttgaatttgatatgaatctttaaaaccagaattggctggatatttcccatgattgggaaaagagtgaaatgaggacattctgtaaactgtccctccctaattccaaggatcagaaactccccgttttgctgactcattccataactggagaaagaagctccattgaccgaagccacagggcagcatggaagtttaaattttctctaaaattaaaatgccaaggataaagctggctgcttccaggagggggaagaggagtggggagtgggcggtgaaacttttccagatgaacggaccataaatgtgttactggctttgtgcctgtagctcattttattatgacctatatgctcctgatttaaagagatctgtgtactgtttacttcccacttcccagaatcccttgtatctcctttctcgggaattgtattttctaataaatgacatttgagaaaaaaatga

SEQ ID NO: 2 - protein, 1634 aa, Homo sapiens
MSSTQGNGEHWKSLESVGISRKELAMAEALQMEYDALSRLRHDKEENRAKQNADPSLISWDEPGVDFYSKPAGRRTDLKLLRGLSGSDPTLNYNSLSPQEGPPNHSTSQGPQPGSDPWPKGSLSGDYLYIFDGSDGGVSSSPGPGDIEGSCKKLSPPPLPPRASIWDTPPLPPRKGSPSSSKISQPSDINTFSLVEQLPGKLLEHRILEEEEVLGGGGQGRLLGSVDYDGINDAITRLNLKSTYDAEMLRDATRGWKEGRGPLDFSKDTSGKPVARSKTMPPQVPPRTYASRYGNRKNATPGKNRRISAAPVGSRPHTVANGHELFEVSEERDEEVAAFCHMLDILRSGSDIQDYFLTGYVWSAVTPSPEHLGDEVNLKVTVLCDRLQEALTFTCNCSSTVDLLIYQTLCYTHDDLRNVDVGDFVLKPCGLEEFLQNKHALGSHEYIQYCRKFDIDIRLQLMEQKVVRSDLARTVNDDQSPSTLNYLVHLQERPVKQTISRQALSLLFDTYHNEVDAFLLADGDFPLKADRVVQSVKAICNALAAVETPEITSALNQLPPCPSRMQPKIQKDPSVLAVRENREKVVEALTAAILDLVELYCNTFNADFQTAVPGSRKHDLVQEACHFARSLAFTVYATHRIPIIWATSYEDFYLSCSLSHGGKELCSPLQTRRAHFSKYLFHLIVWDQQICFPVQVNRLPRETLLCATLYALPIPPPGSSSEANKQRRVPEALGWVTTPLFNFRQVLTCGRKLLGLWPATQENPSARWSAPNFHQPDSVILQIDFPTSAFDIKFTSPPGDKFSPRYEFGSLREEDQRKLKDIMQKESLYWLTDADKKRLWEKRYYCHSEVSSLPLVLASAPSWEWACLPDIYVLLKQWTHMNHQDALGLLHATFPDQEVRRMAVQWIGSLSDAELLDYLPQLVQALKYECYLDSPLVRFLLKRAVSDLRVTHYFFWLLKDGLKDSQFSIRYQYLLAALLCCCGKGLREEFNRQCWLVNALAKLAQQVREAAPSARQGILRTGLEEVKQFFALNGSCRLPLSPSLLVKGIVPRDCSYFNSNAVPLKLSFQNVDPLGENIRVIFKCGDDLRQDMLTLQMIRIMSKIWVQEGLDMRMVIFRCFSTGRGRGMVEMIPNAETLRKIQVEHGVTGSFKDRPLADWLQKHNPGEDEYEKAVENFIYSCAGCCVATYVLGICDRHNDNIMLKTTGHMFHIDFGRFLGHAQMFGNIKRDRAPFVFTSDMAYVINGGDKPSSRFHDFVDLCCQAYNLIRKHTHLFLNLLGLMLSCGIPELSDLEDLKYVYDALRPQDTEANATTYFTRLIESSLGSVATKLNFFIHNLAQMKFTGSDDRLTLSFASRTHTLKSSGRISDVFLCRHEKIFHPNKGYIYVVKVMRENTHEATYIQRTFEEFQELHNKLRLLFPSSHLPSFPSRFVIGRSRGEAVAERRREELNGYIWHLIHAPPEVAECDLVYTFFHPLPRDEKAMGTSPAPKSSDGTWARPVGKVGGEVKLSISYKNNKLFIMVMHIRGLQLLQDGNDPDPYVKIYLLPDPQKTTKRKTKVARKTCNPTYNEMLVYDGIPKGDLQQRELQLSVLSEQGFWENVLLGEVNIRLRELDLAQEKTGWFALGSRSHGTL

SEQ ID NO: 3 - DNA, 7319 bp, Homo sapiens
gagcagaggccctcagggtatagagaaggaagaagagagaggttcactgtagtcctgaagcagaaataagacctgtggctgaaggaagccttagcaattcactccttcctcttcctgagaactctctgtaggaagtctcacctagcagaggcttcacagtatttcagagaagccaaagattgtttgcctctttggaaactgttatccttccatcatgactgtgtcactcctgccactgttccaccatagagatggcgtcctttgcagcaaaccgtaagttataaggatgagggaagaagagtagagggccaaaaggattccattttgaggaaaaactacagtttgccttgccaggtagaagaatcaggcgcccagacaccatgtcacaaccctccagaactgacgttggcaggaagtagagactttgttgcctgtgtcccccatcctcaccatgtcttcgactcagggcaatggggaacactggaagtccctggagtcagtgggcatcagccgcaaagagctagcgatggccgaagccctgcagatggagtatgatgccctgtcccggctccggcatgacaaggaggagaacagagccaagcagaacgcagacccctctctcatcagctgggatgagcctggggtagacttttacagcaagccagcaggaaggcggaccgacctcaagctgttacgcggtctctctggctctgatcctacccttaactacaactcactatccccacaggaagggccgcccaaccactctacctcccaagggccacagcctggctcagatccctggcccaaaggctccctgtctggagactatctctacatttttgatggttcagatgggggagtctcttcgtccccaggaccaggggacatagagggctcttgcaagaaactatccccacctcctctgcctccccgagcttctatctgggatacccctcccctgcctcccagaaaggggtccccctcatcctccaagatctcccagcccagtgacatcaacactttctctttggtcgaacaattgccaggcaaactgctagagcatcggatcctagaagaggaagaggtgctgggaggtgggggtcaggggcgcctactggggtctgtggactatgatggtatcaatgatgcaattactaggctcaacttgaaatcgacctatgatgcggagatgttgcgggatgccaccaggggctggaaggagggccgagggccgctggacttcagcaaagacacctctggaaaacccgtggccaggagcaagactatgccccctcaggtgcccccccgcacctatgcctcccgctatggcaaccgaaagaatgcgacgcctggcaagaaccgccggatttctgcagccccggtgggctcccggccccacactgttgccaatggccatgagttgtttgaggtctcagaagagagagatgaggaggttgctgcattttgccacatgctggatatccttcgatctggctctgacatccaagactacttcctcactggctatgtctggagtgctgtcacccctagcccagagcacctcggggatgaggtcaacctgaaggtgactgtgttgtgtgacaggcttcaagaggcactcactttcacctgcaactgttcctccactgtagacttgcttatctaccagaccctgtgctacacccatgatgacctgaggaatgtggacgtgggtgactttgtgctaaagccctgcgggctggaggagttcctgcagaacaagcatgccttgggcagtcatgagtacatccaatactgccgcaagtttgacattgacattcggctacagctgatggagcagaaggttgtgcgcagtgacctggcccggacggtgaatgatgaccagagcccctccaccttgaactacctcgtccatctccaagagaggcctgtcaagcagaccatcagcaggcaggccctgagtcttctgttcgacacttaccacaatgaggtggatgccttcctgctggctgatggagacttcccactgaaggctgacagggtggtccagtccgtcaaggccatctgcaacgccctggccgccgtggaaacccctgagatcaccagtgctctcaaccagctgcccccctgcccctcccgcatgcagcctaaaattcagaaggatcccagtgtcttggctgtgagggaaaaccgagagaaggtcgtggaagccctgaccgctgccatcttggacctggtggagctgtactgcaacacattcaacgcagacttccagacggcagtgcccgggagccgcaagcatgacctggtccaggaggcctgccatttcgccaggtccctggccttcactgtctatgccacccaccgcatccccatcatctgggctaccagctatgaagatttctacctctcctgctccctcagccatggcggcaaggagctgtgcagccccctgcagacccgaagagctcacttctccaagtacctcttccacctcatcgtctgggaccagcagatctgcttcccagtgcaggtgaaccggctgcctcgggagacactgctgtgtgccactctctatgctctgcccatccccccaccggggagctcctcagaggccaataagcagcggcgggtgcctgaagccctgggctgggtcactaccccactcttcaacttcaggcaggtcctgacctgtggccggaagcttctgggtttgtggccagcaacacaggaaaatcccagcgcccgttggagtgcacctaatttccaccagccagacagtgtcatcctgcagattgacttccccacctcggcctttgacatcaagttcaccagcccccctggagacaagttcagcccccgctatgagtttggcagcctccgggaagaagaccagcgcaagcttaaagacatcatgcagaaagagtccttgtactggctcactgatgctgacaagaagcgcctgtgggagaagcgatattactgccactcggagtggacccacatgaaccaccaggatgccctggggctcctgcatgccaccttcccggaccaggaggtgcgtcgtatggctgtgcagtggattggctcactctcagatgctgagctgctagactacctgccccagctggtacaggccctgaagtatgaatgctacctggacagcccgttggtgcgcttcctcctgaaacgagctgtgtctgacttgagagtgactcactacttcttctggttactgaaggacggcctcaaggactctcagttcagcatccgctaccagtatctgctggcagccttactgtgctgctgtggcaaggggctgagagaagagtttaaccgccagtgctggcttgtcaatgccctggccaaactggcccagcaggtccgggaggcagccccatctgcaaggcagggaatcctccgcacgggcctggaggaggtgaagcagttctttgccctcaatggctcgtgccgcttgccactcagccccagtctgctggttaagggaattgtgcccagggactgttcctacttcaactccaatgctgtccccctcaaactctccttccaaaatgtggatcccctgggtgagaacatccgtgtcatcttcaagtgtggggacgaccttcgccaggacatgctaacgctgcagatgattcgcatcatgagcaagatctgggtccaggaggggctggacatgcgcatggtcatcttccgctgcttctccaccggccggggcagagggatggtggagatgatccctaatgctgagaccctgcgtaagatccaggtggagcatggggtgaccggctcgttcaaggaccggcccctggcagactggctgcagaaacacaaccctggggaggacgagtatgagaaggctgtggagaactttatctactcctgcgctggctgctgcgtggccacgtacgtcttgggcatctgtgaccgacataatgacaacatcatgctgaagaccactggtcacatgttccacattgattttggccgcttcctgggccatgcccagatgtttggcaacatcaagcgggaccgtgccccctttgtcttcacctcggacatggcgtatgtcatcaacgggggtgacaagccttccagccgcttccatgattttgttgacctttgctgccaagcctacaacctcattcgcaagcacacccacctcctcctcaaccttctgggcctgatgttgtcctgtgggatccctgaactctcagacctggaggacctcaagtatgtgtacgatgccctgaggcctcaggatacagaggccaatgccactacctacttcactaggttgattgagtccagcctgggcagtgtagccacaaagctcaattttttcatccataatctggctcagatgaagttcacgggctcagatgaccggctgaccctctcctttgcctcccgaacacacactctcaagagctctggccgaatcagtgatgttttcctctgccgccatgagaagatcttccaccccaacaaaggctatatatatgtggtaaaggtgatgcgagagaacactcacgaggccacctacatccagcggacctttgaggagttccaggaattacacaataagttgcggctgctcttcccttcttcccacttgcccagcttccctagtcgcttcgtgatcggccgctcccggggagaggcggtggccgagcggcggagggaggagctaaacggttacatctggcacttgatccacgcaccccctgaggtggccgagtgtgatttggtgtacaccttcttccacccactgccccgggatgagaaggctatgggcaccagcccagctcctaagtcctcagatggcacatgggcccggcccgtcggaaaggtgggaggggaggtgaagctgtccatctcctacaaaaacaataaactcttcatcatggtgatgcatattcggggcttgcaactgctccaggatggaaatgaccctgacccctatgtgaaaatttacctccttcctgaccctcagaaaaccactaagaggaaaaccaaagtggcccggaaaacctgcaatcctacctacaatgagatgttggtatatgatgggatccccaagggtgacctgcagcagcgggagctccagctgagcgtgctgagtgagcagggattctgggagaacgtcctcctcggtgaggtgaacatccgcctgcgagagctggacctggctcaggagaagaccggctggttcgccctgggatctcgaagtcatggcaccttgtgagcccagcagagccaccacccagcatcccaggctggtggcaggagctgggggagaggactctcccctgtgagactcctccttgtgaagggccagggccctgggcaggcctccagctcggtccaggtgattctggcctctgtggtaggaggcagggagagtaagacatgctctgctgtctcttcctctggagactgaacttgggttggttgtgatgagcagccccttggaggctgtgaggttgcagcaaagttttaagtttaccttgtgtcaagggagcaatgcttggtttggggagtgtgtggggtgggctgtatgaagtagcattttgggggtgggtgggtggatatcttaatttttatttttaaaaaatgaaatagtgatgttgtcctaactgggacaggaagccttgcgagaagggacgtacctatgccccagaaggcaagagaggaagactatttggactttttgtatgattaaggttcttattggacttttccctaggttttttttttttgttattgttgttgttgtttcgttttctagctataggaactatctggggaggggcccagtgggtcctcggccagagccctctctaaggacaggttggggagggttggggagggctgcctgtgctggactgaggcttgtgcctctgggcctttctgattttgcctccaaaggagagcgctgtgatacctacatgtgtaaggaagggccttccgtattggggttctgccaaggacccgtattcagggacccatgctcttttggggggacttttcctcttgtcttccctactttattaggacttgccctgaataccattttctaccccttgcccctccattctcctggcccttctgggggtcagctggtctctatgaatatgctgggggtgcttccccataggtctctcccttcatttgtctctggtgggacaaaatactgactcagtccttagatgtagtttcacccaagagcatcttggccctgggaagaggtccctaggctgcagatgctactgactgcttgctaggtagcctctggaaagcattccccatccatcactccccacttctttctgctgtgctgcttccctcccaaactccatttctgtcaccctttttataagacttttcctcattctgtggggccataaacctatttagtctggagccaaagggatgccctatctgaaggaaaggggcatggggtgggggattccatcaaaactgttgttttttgccccatgatttttctttggtcagtaggaggctggattggagtggtgattattcccctggagctaagctcaggagcccgaagggagagactgagactgactcccttatctcttcatattctttattccctaccagatggattttttttttttttttttggagacggagtctcgccctgtcgccaggctggagtgtagtggcatgatctcgactcactgcaaaatctgcctcccgggttcaagcgattctcctacctcagcctcccgagtagctgggattacaggcatgtgccaccacgccaagctaatttttgtatttttagtagagacggggtttcaccatgttggccaggatggtctcgatctcttgacctcgtgatctgcctgccttggcctcccaaagtgctgggattacaggcgtgagccaccatgccccgccccagatggattttacatttgctcttttgtgtttcgctccaaagggttgtcttcctcgccaaaaggagggagggactttgaatttgatatgaatctttaaaaccagaattggctggatatttcccatgattgggaaaagagtgaaatgaggacattctgtaaactgtccctccctaattccaaggatcagaaactccccgttttgctgactcattccataactggagaaagaagctccattgaccgaagccacagggcagcatggaagtttaaattttctctaaaattaaaatgccaaggataaagctggctgcttccaggagggggaagaggagtggggagtgggcggtgaaacttttccag

SEQ ID NO: 4 - protein, 1606 aa, Homo sapiens [sequence omitted]

SEQ ID NO: 5 - DNA, 21 bp, Artificial Sequence
  Synthetic oligonucleotide
ccaagaucucucagccuaatt

SEQ ID NO: 6 - RNA, 21 nt, Artificial Sequence
  Synthetic oligonucleotide
uuaggcugagagaucuuggag

SEQ ID NO: 7 - DNA, 21 bp, Artificial Sequence
  Synthetic oligonucleotide
gggugguccagucugucaatt

SEQ ID NO: 8 - DNA, 21 bp, Artificial Sequence
  Synthetic oligonucleotide
uugacagacuggaccaccctg

SEQ ID NO: 9 - DNA, 1820 bp, Homo sapiens
gggtttacgctgccgccggcatccgctcggacgcggccacgttgtcttgcgcgctttgcccgcctggccctgggactctgaccctcggctaccctttcctgccccactagcgtggccgcgagcctcggtgagccggccgtattcccgctctcgcttagggggcacaggcgcaggcatcggcccggccactccaagccttcggtgcgcgggcgcgtctgggatacgggcccgggaggcgccgccctccgtccgcccggtgcctctcaggaacagcgaaccggagagagcgccggagagttgggctcagtgcagagctcggcgccggggcccatgcccgtgcgcccccgcaggccggcgccatggcctccgggagtgtggccgagtgcctgcagcaggagaccacctgccccgtgtgcctgcagtacttcgcagagcccatgatgctcgactgcggccataacatctgttgcgcgtgcctcgcccgctgctggggcacggcagagactaacgtgtcgtgcccgcagtgccgggagaccttcccgcagaggcacatgcggcccaaccggcacctggccaacgtgacccaactggtaaagcagctgcgcaccgagcggccgtcggggcccggcggcgagatgggcgtgtgcgagaagcaccgcgagcccctgaagctgtactgcgaggaggaccagatgcccatctgcgtggtgtgcgaccgctcccgcgagcaccgcggccacagcgtgctgccgctcgaggaggcggtggagggcttcaaggagcaaatccagaaccagctcgaccatttaaaaagagtgaaagatttaaagaagagacgtcgggcccagggggaacaggcacgagctgaactcttgagcctaacccagatggagagggagaagattgtttgggagtttgagcagctgtatcactccttaaaggagcatgagtatcgcctcctggcccgccttgaggagctagacttggccatctacaatagcatcaatggtgccatcacccagttctcttgcaacatctcccacctcagcagcctgatcgctcagctagaagagaagcagcagcagcccaccagggagctcctgcaggacattggggacacattgagcagggctgaaagaatcaggattcctgaaccttggatcacacctccagatttgcaagagaaaatccacatttttgcccaaaaatgtctattcttgacggagagtctaaagcagttcacagaaaaaatgcagtcagatatggagaaaatccaagaattaagagaggctcagttatactcagtggacgtgactctggacccagacacggcctaccccagcctgatcctctctgataatctgcggcaagtgcggtacagttacctccaacaggacctgcctgacaaccccgagaggttcaatctgtttccctgtgtcttgggctctccatgcttcatcgccgggagacattattgggaggtagaggtgggagataaagccaagtggaccataggtgtctgtgaagactcagtgtgcagaaaaggtggagtaacctcagccccccagaatggattctgggcagtgtctttgtggtatgggaaagaatattgggctcttacctccccaatgactgccctacccctgcggaccccgctccagcgggtggggattttcttggactatgatgctggtgaggtctccttctacaacgtgacagagaggtgtcacaccttcactttctctcatgctaccttttgtgggcctgtccggccctacttcagtctgagttactcgggagggaaaagtgcagctcctctg

SEQ ID NO: 10 - protein, 513 aa, Homo sapiens
MASGSVAECLQQETTCPVCLQYFAEPMMLDCGHNICCACLARCWGTAETNVSCPQCRETFPQRHMRPNRHLANVTQLVKQLRTERPSGPGGEMGVCEKHREPLKLYCEEDQMPICVVCDRSREHRGHSVLPLEEAVEGFKEQIQNQLDHLKRVKDLKKRRRAQGEQARAELLSLTQMEREKIVWEFEQLYHSLKEHEYRLLARLEELDLAIYNSINGAITQFSCNISHLSSLIAQLEEKQQQPTRELLQDIGDTLSRAERIRIPEPWITPPDLQEKIHIFAQKCLFLTESLKQFTEKMQSDMEKIQELREAQLYSVDVTLDPDTAYPSLILSDNLRQVRYSYLQQDLPDNPERFNLFPCVLGSPCFIAGRHYWEVEVGDKAKWTIGVCEDSVCRKGGVTSAPQNGFWAVSLWYGKEYWALTSPMTALPLRTPLQRVGIFLDYDAGEVSFYNVTERCHTFTFSHATFCGPVRPYFSLSYSGGKSAAPLIICPMSGIDGFSGHVGNHGHSMETSP

(19) United States
(12) Patent Application Publication
(10) Pub. No.: US 2015/0204846 A1
(43) Pub. Date: Jul. 23, 2015

(54) PHOSPHATIDYLINOSITOL-3-KINASE C2 BETA MODULATORS AND METHODS OF USE THEREOF

(71) Applicant: NEW YORK UNIVERSITY, New York, NY (US)

(72) Inventors: Edward Skolnik, New York, NY (US); Zhai Li, Rego Park, NY (US); Shekhar Srivastava, Millburn, NJ (US)

(21) Appl. No.: 14/400,985

(22) PCT Filed: May 15, 2013
(86) PCT No.: PCT/US2013/041137

§ 371 Date: Nov. 13, 2014

Related U.S. Application Data
(60) Provisional application No. 61/647,189, filed on May 15, 2012.

(51) Int. Cl. G01N 33/50; C12N 15/113

(52) U.S. Cl. CPC G01N 33/502; C12N 15/1137; G01N 33/5047; C12N 2310/14; G01N 2333/91215; G01N 2500/10; G01N 2500/04

(57) ABSTRACT

Methods for screening to identify agents capable of modulating PI3KC2β/mast cell activation are also encompassed herein as are methods of using such agents to treat IgE-mediated allergic disorders.

### CROSS REFERENCE TO RELATED APPLICATIONS

This application claims priority under 35 USC §119(e) from U.S. Provisional Application Ser. No. 61/647,189, filed May 15, 2012, which application is herein specifically incorporated by reference in its entirety.

### GOVERNMENTAL SUPPORT

The research leading to the present invention was funded in part by NIH grants R01GM084195 and R01AI052459. The United States government may have certain rights in the invention.

### FIELD OF THE INVENTION

The present invention pertains to the fields of immunology, allergic reactions, and mast cell activity. More particularly, the invention relates to in vitro screening methods directed to identifying agents capable of modulating mast cell activity and in vitro and in vivo methods directed to modulating mast cell activity. As described herein, agents that inhibit class II phosphatidylinositol-3-kinase C2 beta (PI3KC2β) are envisioned as exemplary modulators (i.e., inhibitors) of mast cell activity and may be used to advantage as therapeutic agents for treating IgE mediated allergic disorders.

### BACKGROUND OF THE INVENTION

Several publications and patent documents are referenced in this application in order to more fully describe the state of the art to which this invention pertains. The disclosure of each of these publications and documents is incorporated by reference herein.

Mast cells play an important role in IgE mediated allergic reactions such as allergic rhinitis, anaphylaxis, asthma and immediate type hypersensitivity. Binding of IgE to the high affinity IgE receptor, FcεR1, on mast cells triggers receptor oligomerization and activation (2, 14). Activation then results in the immediate release of preformed mediators including histamine, proteases, and a number of cytokines that are stored in cytoplasmic granules. In addition, activation of FcεR1 results in the de novo synthesis of a number of proinflammatory cytokines and lipids.

Ca²⁺ functions as a critical second messenger to mediate both degranulation and the production of proinflammatory cytokines (9, 11, 15). Crosslinking of FcεR1 activates phospholipase Cγ resulting in the generation of inositol-1,4,5-trisphosphate (IP3), which binds its receptor on the endoplasmic reticulum leading to the release of Ca²⁺ into the cytoplasm. This in turn results in the oligomerization of STIM1 and its subsequent association with and activation of the calcium-release activated Ca²⁺ (CRAC) channels at the plasma membrane, which is the predominant mechanism for Ca²⁺ influx into mast cells and other immune cells (8, 16, 24, 25, 36). The important role for this pathway in mast cells is supported by the findings that FcεR1 stimulated degranulation is markedly defective in bone marrow derived mast cells (BMMC) derived from Stim1−/− and CRAC1M−/− mice (1, 35). In addition, FcεR1 induced in vivo anaphylaxis is markedly inhibited in CRAC1M−/− mice (35).

The influx of Ca²⁺ into mast cells via CRAC channels is dependent on maintaining a negative membrane potential, which provides the electrical driving force for rapid Ca²⁺ influx. Previous studies have shown that the intermediate conductance Ca²⁺-activated K⁺ channel, KCa3.1 (also known as SK4 and KCNN4), via the efflux of K⁺, is critical for maintaining a negative membrane potential and is required for maximal FcεR1 stimulated Ca²⁺ influx and degranulation in mast cells (19, 27). On the other hand, activation of the Ca²⁺-activated nonselective transient receptor potential Melastatin 4 (TRPM4) channel functions to depolarize the membrane potential and limit FcεR1 stimulated Ca²⁺ influx (34).

KCa3.1 also plays a critical role in Ca²⁺ flux and cytokine production following T cell receptor (TCR) activation (10, 30). Previously, it has been shown that TCR stimulation results in the activation of PI3KC2β leading to the generation of phosphatidylinositol 3 phosphate (PI3P), which is required for the histidine kinase, nucleoside diphosphate kinase B (NDPKB), to phosphorylate and activate KCa3.1 (29, 30). A role for PI3KC2β in KCa3.1 activation is supported by the findings that siRNA knockdown of PI3KC2β results in decreased KCa3.1 channel activity and TCR stimulated Ca²⁺ flux and cytokine production, while these same responses are all increased by T cells overexpressing PI3KC2β (29).

The present inventors have, moreover, recently found that the tripartite motif containing protein, TRIM27 negatively regulates KCa3.1 channel activity and TCR-stimulated Ca²⁺ influx and cytokine production in activated CD4 T cells by functioning as an E3 ligase (4).

### SUMMARY OF INVENTION

Cross linking of the IgE receptor (FcεR1) on mast cells plays a critical role in IgE-dependent allergy, examples of which include allergic rhinitis, asthma, anaphylaxis, and immediate type hypersensitivity reactions. Previous studies have demonstrated that the K⁺ channel, KCa3.1, plays a critical role in IgE-stimulated Ca2+ entry and degranulation in both human and mouse mast cells. Evidence presented herein demonstrates that the class II phosphatidylinositol 3 kinase C2β (PI3KC2β) is required for FcεR1 stimulated activation of KC3.1 and Ca²⁺ influx in BMMC. The present inventors have also determined that the E3 ubiquitin ligase, TRIM27, negatively regulates FcεR1 stimulated activation of KCa3.1, Ca²⁺ influx, degranulation, and production of cytokines by BMMC by ubiquitinating and inhibiting PI3KC2β. In further support of this functional relationship, TRIM27−/− mice are also more susceptible to passive anaphylaxis. Accordingly, the present findings identify TRIM27 as an important negative regulator of mast cells in vivo, and suggest that PI3KC2β is a potential new pharmacologic target to treat IgE mediated disease.

In one aspect, the present invention is directed to a method for screening to identify an inhibitor of phosphatidylinositol-3-kinase C2 beta (PI3KC2β) activity, the method comprising: contacting PI3KC2β or a functional fragment thereof with at least one candidate agent of a plurality of candidate agents and measuring PI3KC2β activity in the presence of the at least one candidate agent, wherein a reduction or inhibition of PI3KC2β activity in the presence of the at least one candidate agent relative to that measured in the absence of a candidate agent or presence of a control agent identifies the at least one candidate agent as the inhibitor of PI3KC2β activity.

Exemplary PI3KC2β functional fragments comprise the kinase domain and larger fragments comprising the kinase domain of the full-length protein. Exemplary functional fragments comprise or consist of amino acids spanning 987-1340, which encompass the kinase domain, of the full-length protein. Nucleic and amino acid sequences (SEQ ID NOs: 1 and 2) of human phosphatidylinositol-4-phosphate 3-kinase, catalytic subunit type 2 beta (PIK3C2B) are presented herein. See also NCBI Reference Sequence: NM_{—}002646.3, the entire content of which is incorporated herein by reference. Nucleic and amino acid sequences (SEQ ID NOs: 3 and 4) of human phosphoinositide-3-kinase, class 2, beta polypeptide, mRNA (cDNA clone MGC:177879 IMAGE:9052862), complete coding DNA sequence (cds) are also presented herein. See also NCBI Reference Sequence: BC144342, the entire content of which is incorporated herein by reference.

In an embodiment of the method, the plurality of candidate agents comprises a library. In a more particular embodiment, the library is a small molecule or chemical library.

As described herein, the contacting can be performed in a vessel or in cell culture. In a particular embodiment, the vessel is a test tube or a well of a multi-well plate. Such a vessel comprises a solution or buffer that is compatible with PI3KC2β activity. Solutions and buffers compatible with PI3KC2β activity are aqueous and satisfy the pH and compositional criteria required for full kinase activity.

In a further embodiment, a secondary screen is envisioned, whereby the inhibitor of PI3KC2β activity identified in a primary screen is assessed with respect to its ability to inhibit other kinases. Such kinases include the class I phosphatidylinositol-3-kinase (PI3K) p110α, the epidermal growth factor receptor kinase, ERK map kinase, or Janus Kinase 3. Secondary screens are utilized to ensure that the inhibitor of PI3KC2β activity identified in the primary screen is specific for PI3KC2β.

In embodiments wherein the primary method is performed in a vessel, the method may further comprise assessing the ability of the inhibitor of PI3KC2β activity identified a vessel (e.g., a test tube) to inhibit PI3KC2β activity in cells in cell culture or in a subject. Cells used in such secondary screens in cell culture may be mast cell lines or primary mast cells derived from or generated from bone marrow or peripheral blood in vitro. Mast cells generated from bone marrow are referred to herein as bone marrow derived mast cells (BMMCs). Exemplary mast cell lines include, without limitation, the following: the MC-9 cell line and the HC-1 cell line.

In embodiments wherein a secondary screen is performed in a subject, the subject may be a mammal afflicted with an IgE-mediated allergic disorder. Exemplary IgE-mediated allergic disorders include, without limitation, allergic rhinitis, allergic or atopic asthma, anaphylaxis, atopic dermatitis, eczema, hay fever, fibromyalgia, and an immediate type hypersensitivity reaction.

In a further aspect, a method for treating a subject afflicted with an IgE-mediated allergic disorder is described, the method comprising administering a therapeutically effective amount of the inhibitor of PI3KC2β activity identified using methods described herein or a composition thereof to the subject, wherein the administering confers relief from symptoms of the IgE-mediated allergic disorder, thereby treating the subject. Also encompassed herein is the use of an inhibitor of PI3KC2β activity identified using methods described herein or a composition thereof to treat a subject afflicted with an IgE-mediated allergic disorder comprising administering a therapeutically effective amount of the inhibitor of PI3KC2β activity to the subject to confer relief from symptoms of the IgE-mediated allergic disorder, thereby treating the subject. Also envisioned herein is the use of an inhibitor of PI3KC2β activity identified using methods described herein or a composition thereof in the preparation of a medicament for the treatment of an IgE-mediated allergic disorder. An IgE-mediated allergic disorder with which the subject is afflicted may be, without limitation, allergic rhinitis, asthma, anaphylaxis, or an immediate type hypersensitivity reaction. Symptoms of allergic rhinitis include, for example, excess nasal secretion, itching and nasal obstruction. Symptoms of asthma include, for example, airway obstruction, wheezing, and shortness of breath. Symptoms of anaphylaxis include, for example, decreased blood pressure, respiratory failure with bronchoconstriction, and skin rash due to release of mediators from cells such as mast cells. Symptoms of an immediate type hypersensitivity reaction include, for example, a drop in blood pressure, bronchoconstriction, itching, and inflammation of the gastrointestinal tract. It is, therefore, envisioned that administration of an inhibitor of PI3KC2β activity or a composition thereof to a subject in need thereof would confer symptomatic relief to the subject with respect to at least one of the aforementioned symptoms of the particular IgE-mediated allergic disorder with which the subject is afflicted. In a particular embodiment, the subject is a mammal. In a more particular embodiment, the mammal is a human.

Other features and advantages of the invention will be apparent from the following description of the particular embodiments thereof, and from the claims.

### BRIEF DESCRIPTION OF THE DRAWINGS

FIG. 1. Generation of BMMCs from TRIM27^{+/+} and TRIM27^{-/-} mice. (A) Lysates of TRIM27^{+/+} and TRIM27^{-/-} BMMCs immunoblotted with antibodies to TRIM27 and PI3KC2β. (B) FACS analysis demonstrating similar levels of expression of FcεR1 on TRIM27^{+/+} and TRIM27^{-/-} BMMCs.

FIG. 2. PI3KC2β is required for FcεR1 stimulated activation of KCa3.1 and Ca²⁺ influx of BMMCs. (A) Real time PCR of PI3KC2β in TRIM27^{+/+} BMMC transfected with a control or siRNA to PI3KC2β. (B) TRIM27^{+/+} BMMC transfected with (i) siRNA to PI3KC2β were sensitized with anti-DNP IgE, and whole cell patch clamp was performed with (b) or without (a) stimulation of FcεR1 with DNP-HSA. (ii) To verify that the decrease in KCa3.1 channel activity in siRNA PI3KC2β transfected cells was due to the decreased levels of PI3P, rescue of channel activity was assessed after addition of PI3P (100 nM) to the pipette solution during patch clamping of siRNA transfected cells. (C) Bar graph summary of whole-cell patch-clamp experiments performed in B. Also shown is current from another siRNA to PI3KC2β (2) and the failure of PI(4)P and PI(3,4,5)P₃ to rescue KCa3.1 channel activity in PI3KC2β 1 siRNA transfected cells. All experiments shown are representative of at least three experiments performed on cells isolated from three separate mice. *p<0.05 as compared to the current in TRIM27^{+/+} BMMC or as indicated. (D) Mast cells were loaded with Fura-2 AM (5 mM) and Ca²⁺ flux was determined after cross-linking with DNPHSA as described in B.

FIG. 3. Increased KCa3.1 channel activity and FcεR-stimulated Ca²⁺ influx in TRIM27^{-/-} BMMCs. (A) TRIM27^{+/+} (i) and TRIM27^{-/-} (ii) BMMCs were senstitized with anti-DNP IgE and KCa3.1 channel activity was assessed before (a) and after (b) stimulation with DNP-HSA. (B) Bar graph summary of results in (A) at +40 mV (n=10 cells). *p<0.05 as compared to the current in TRIM27^{+/+} BMMC. BMMC isolated from TRIM27^{-/-} mice were either transfected with a control siRNA or a siRNA to PI3KC2β and (C) whole cell patch and (D) Ca²⁺ influx was assessed as indicated in FIG. 2 (D). Also shown in (C) is rescue of KCa3.1 channel activity in PI3KC2β siRNA transfected cells by the addition of PI3P to the pipette solution. *p<0.05 as compared to the current in TRIM27^{-/-} BMMCs or as indicated. (E) Cells were sensitized as in A, loaded with Fura2 AM, and FcεR stimulated Ca²⁺ influx was assessed following stimulation with DNP-HSA. (F) TRIM27^{+/+} and TRIM27^{-/-} BMMCs were activated as above and change in membrane potential was determined. *p<0.05 as compared to the membrane potential measured in TRIM27+/+ BMMC.

FIG. 4. β-hexosaminadase release and cytokine production is increased in TRIM27^{-/-} BMMCs. (A) 1×10⁶ TRIM27^{+/+} and TRIM27^{-/-} BMMCs were plated into 96 well plates, senstitized with anti-DNP IgE, and then stimulated with various concentrations of DNP-HSA for 30 minutes. Shown is the release of β-hexosaminadase into the supernatants after correcting for spontaneous release. *p<0.05 as compared to the release measured in WT at the same concentration. (B) Cells were stimulated as in A for various periods of time and induction of (i) TNFα, (ii) IL-6, and (iii) IL13 mRNA was assessed by RTPCR. *p<0.05 as compared to the mRNA levels in WT at the same time point.

FIG. 5. Systemic anaphylaxis in TRIM27^{+/+} and TRIM27^{-/-} mice. (A) Mean decrease in body temperature (° C.) of TRIM27^{+/+} and TRIM27^{-/-} mice following induction of anaphylaxis (n=5 mice in each group). (B) Mean serum histamine levels 30 minutes after induction of anaphylaxis (n=5 mice in each group). *p<0.05 as compared to the WT.

FIG. 6. FcεR stimulated tyrosine phosphorylation of proximal signaling molecules and the ERK Map kinase pathway is similar between TRIM27^{+/+} and TRIM27^{-/-} BMMCs. Lysates from TRIM27^{+/+} and TRIM27^{-/-} BMMCs were stimulated with DNP-HSA for various lengths of time following sensitization with DNP-IgE and then immunoblotted with anti-phosphospecific antibodies as indicated.

FIG. 7. PI3KC2β knockout mice are less susceptible to passive cutaneous anaphylaxis. The histogram bar graph shows PI3KC2β knockout mice exhibit an impaired response to passive cutaneous anaphylaxis relative to wild type control littermates as assessed by decreased extravasation of Evan's blue dye, which serves as an indicator of leaky capillaries.

FIG. 8. Systemic anaphylaxis in PI3K-C2beta⁺/+ (WT) and PI3K-C2beta⁻/⁻ mice. (A) Mean decrease in body temperature (° C.) of PI3K-C2beta⁺/+ and PI3K-C2beta⁻/− mice following induction of passive systemic anaphylaxis (n=5 mice in each group). (B) Mean serum histamine levels 30 min after induction of anaphylaxis (n=5 mice in each group). *, P<0.05 as compared to results for the WT. (C) Passive cutaneous anaphylaxis in PI3K-C2beta⁺/+ and PI3K-C2beta⁻/− mice. Data are expressed as A₆₂₀ per g of skin (n=5 to 8 mice in each group). *, P<0.05 compared to results for the WT or as indicated.

### DETAILED DESCRIPTION OF THE INVENTION

Mast cells play a critical role in IgE-dependent allergy including allergic rhinitis, asthma, anaphylaxis, and immediate type hypersensitivity reactions. The results presented herein demonstrate that IgE activation of mast cells in vivo and in vitro requires the activation of the class 2 phosphatidylinositol 3 kinase (PI3K), PI3KC2β. In light of these results, the present inventors propose that drugs that inhibit PI3KC2β will block activation of mast cells and thereby provide a novel mechanism to treat IgE mediated diseases such as asthma and allergy.

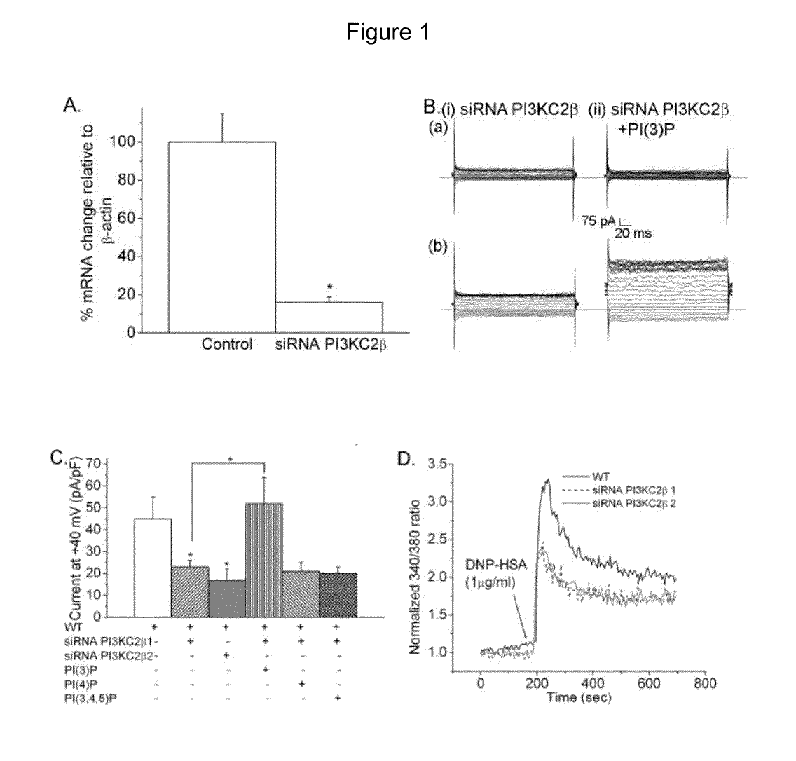

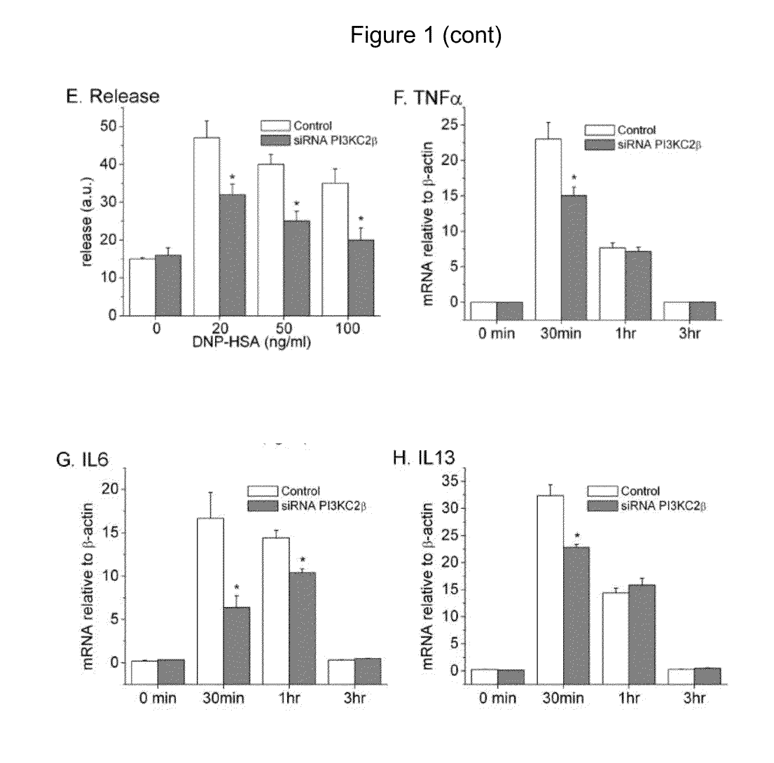

Results presented herein reveal that PI3KC2β activation is required for activation of the potassium channel KCa3.1 and calcium (Ca²⁺) influx in BMMCs. By way of background, influx of Ca²⁺ into mast cells is critical for mast cell activation, and is directly responsible for mast cell production of inflammatory cytokines as well as the exocytosis of intracellular mediators of inflammation such as histamine. The present inventors show herein that PI3KC2β is required for IgE-stimulated Ca²⁺ influx into mast cells, which is mediated via PI3KC2β activation of the potassium channel KCa3.1. This is supported by the present findings that siRNA knockdown of PI3KC2β results in decreased IgE-stimulated KCa3.1 channel activity and Ca²⁺ influx into mast cells (see, for example, FIG. 1A-D). This is, moreover, associated with a decrease in IgE-stimulated induction of inflammatory cytokines and release of intracellular inflammatory mediators (see, for example, FIGS. 1A and E-H).

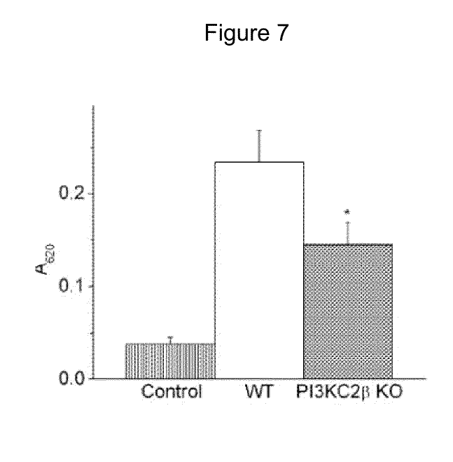

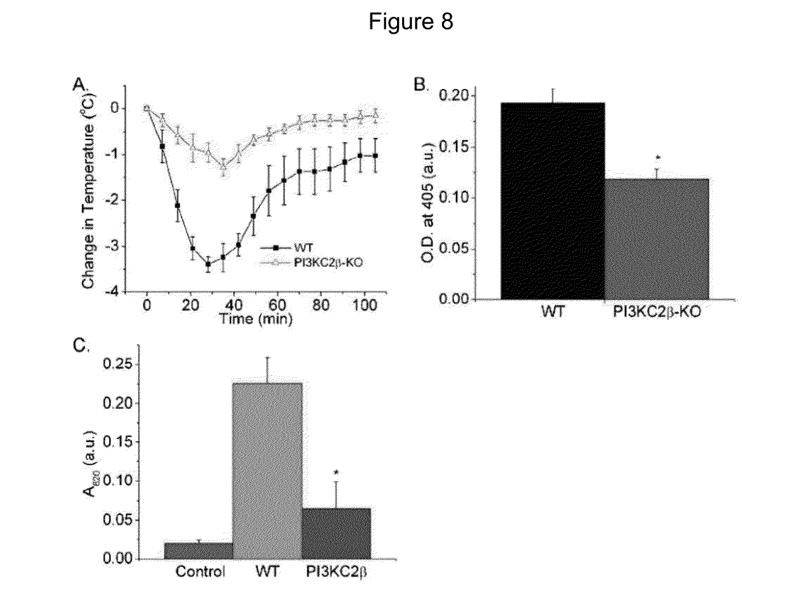

Results presented herein also reveal that PI3KC2β knockout mice are less susceptible to passive cutaneous anaphylaxis. To assess whether changes in mast cells in vitro are also relevant in vivo, PI3KC2β knockout or littermate control wild type mice were assessed for their response to passive cutaneous anaphylaxis. Mice were sensitized intradermally with anti-DNP IgE and 24 hours later were injected intravenously with DNP-HSA containing 0.5% Evan's blue dye. Thirty minutes after dye injection, mice were sacrificed, and tissue sections around the intradermal injection site excised and weighed, followed by extraction of extravasated Evan's blue dye by incubation of biopsies in 0.5 ml formamide at 55° C. for 24 h and measurement of absorbance at 620 nm. These results demonstrated that PI3KC2β knockout mice had an impaired response to passive cutaneous anaphylaxis as assessed by decreased extravasation of dye indicating less leaky capillaries (see FIG. 7). Results presented in FIG. 8 corroborate those of FIG. 7 with regard to the impaired response to passive cutaneous anaphylaxis and, furthermore, reveal that PI3KC2β knockout mice had an impaired response to passive systemic anaphylaxis. Thus, these findings indicate that mast cells in vivo in PI3KC2β knockout mice are less sensitive to degranulation following antigen stimulation.

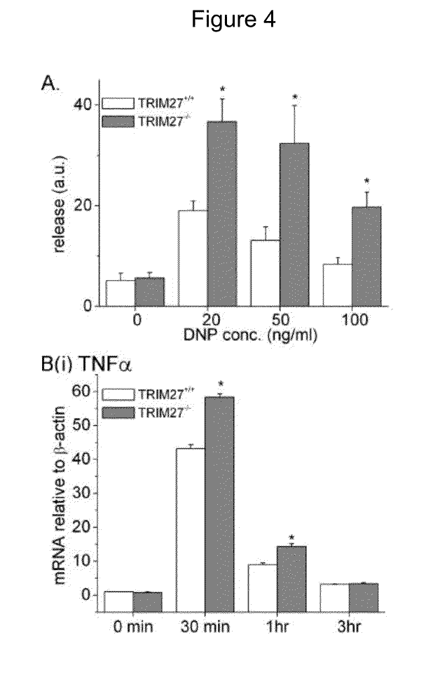

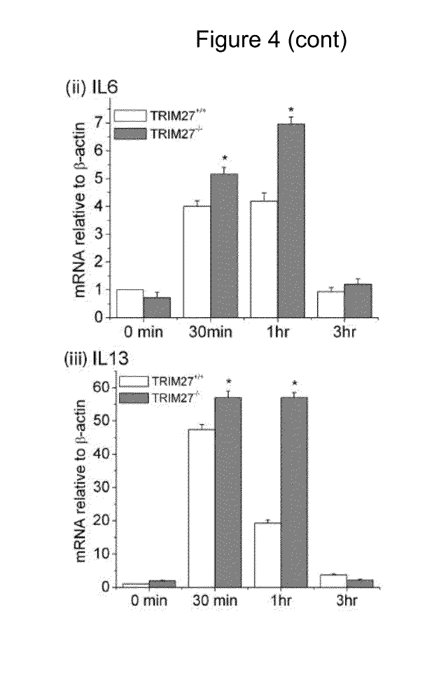

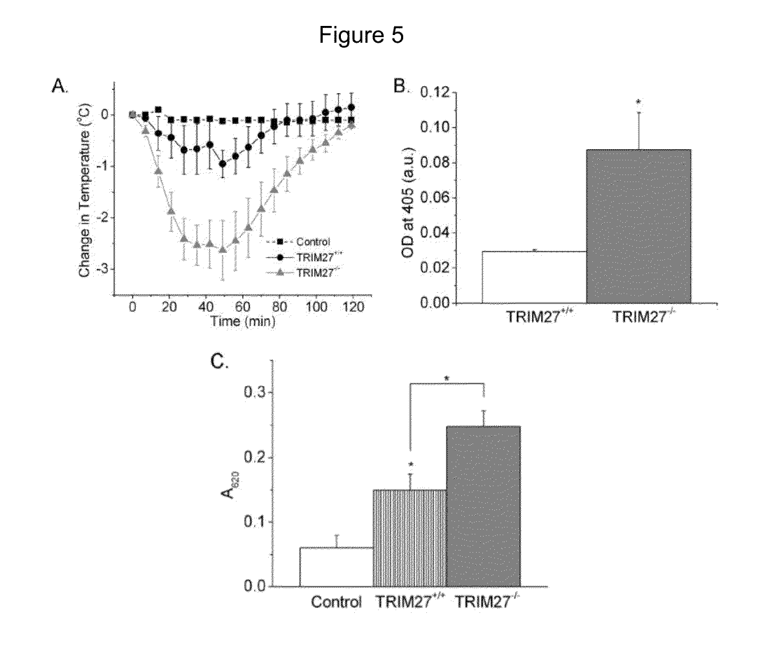

On the flip side, we also identified a new negative regulator of PI3KC2β, tripartite motif containing protein 27 (TRIM27). Consistent with a critical role for PI3KC2β in mast cell activation, we found that mast cells isolated from TRIM27 knockout mice had increased PI3KC2β kinase activity, which resulted in an increase in KCa3.1 channel activity and IgE-stimulated production of cytokines and inflammatory mediators (see, for example, FIG. 4). In addition, TRIM27 knockout mice were more susceptible in vivo to passive systemic and cutaneous anaphylaxis, which was associated with the increased production of blood histamine levels (see, for example, FIG. 5).

The findings presented herein, therefore, provide a strong rationale to screen for and develop drugs that inhibit PI3KC2β activity with the prediction that such drugs can be used advantageously to treat IgE mediated allergy and asthma. As described herein below, screens for PI3KC2β inhibitors can be performed using high-throughput assays and available chemical libraries.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which is placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4,7,2′,7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More particularly, the preparation comprises at least 75% by weight, and most particularly 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, viral transduction, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v).

The term “about” as used herein refers to a variation in a stated value or indicated amount of up to 10%, 9%, 8%, 7%, 6%, 5%, 4%, 3%, 2%, 1%, 0.75%, 0.5%, 0.25% or 0.1%., wherein the variation can be either an increase or a decrease in the stated value or indicated amount. Use of the term may, therefore, be used to establish a range of values or amounts.

As used herein, the term “mast cell” refers to a bone marrow derived cell that mediates hypersensitivity reactions. Mast cells are characterized by the presence of cytoplasmic granules (histamine, chondroitin sulfate, proteases) that mediate hypersensitivity reactions, high levels of the receptor for IgE (IgEεRI), and require stem cell factor (cytokine) for development. Mature mast cells are not found in the circulation, but reside in a variety of tissues throughout the body.

As used herein, the term “bone marrow cell derived mast cell” refers to a mast cell derived in vitro from bone marrow hematopoietic stem cells.

As used herein, the term “anaphylaxis” refers to a life threatening allergic reaction characterized by decreased blood pressure, respiratory failure with bronchoconstriction, and skin rash due to release of mediators from cells such as mast cells. See also FIGS. 7 and 8.

As used herein, the term “a solution compatible with PI3KC2β activity” refers to solution or buffer in which PI3KC2β retains its activity, namely the ability of PI3KC2β to transfer phosphate from adenosine triphosphate (ATP) to phosphatidylinositol.

As used herein, the term “IgE-mediated allergic disorder” refers to allergic disorders mediated by binding of an IgE antibody to its receptor on mast cells, resulting in mast cell activation

As used herein, the term “allergic rhinitis” refers to allergic inflammation of the nasal airways resulting in excess nasal secretion, itching and nasal obstruction. This condition is frequently mediated by IgE antibodies to pollen which subsequently activate mast cells.

As used herein, the term “asthma” refers to an inflammatory disease of the respiratory airways that is characterized by airway obstruction, wheezing, and shortness of breath.

As used herein, the term “immediate type hypersensitivity reaction” refers to an acute allergic response to an allergen that is characterized by a drop in blood pressure, bronchoconstriction, itching, and inflammation of the gastrointestinal tract. Exemplary immediate type hypersensitivity reactions include: reactions to drugs, insect venoms, pollens, and food.

As used herein, the term “modulator” refers to a compound or molecule that is capable of altering an activity such that the activity is either inhibited/decreased or enhanced/increased in the presence of the modulator relative to the level of activity in the absence of the modulator or in the presence of a negative control compound. Modulators can, therefore, either be inhibitors or enhancers of the activity being measured. With respect to the present screening methods, the presence of an inhibitor of PI3KC2β decreases or inhibits PI3KC2β activity.

As used herein, the term “candidate compound” or “test compound” refers to any compound or molecule that is to be tested. As used herein, the terms, which are used interchangeably, refer to biological or chemical compounds such as simple or complex organic or inorganic molecules, peptides, proteins, peptidomimetics, peptide mimics, antibodies, nucleic acids (DNA or RNA), oligonucleotides, polynucleotides, antisense molecules, small interfering nucleic acid molecules, including siRNA or snRNA, carbohydrates, lipoproteins, lipids, small molecules and other drugs. In a particular embodiment, the siRNA or snRNA targets PI3KC2β. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed.

As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action. Moreover, the agent may be selected by its effect on the gene expression profile obtained from screening in vitro or in vivo. Furthermore, candidate compounds can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (now U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

If the screening for compounds that modulate the expression, activity or function of PI3KC2β is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan. In addition, a secondary screen may include assessing the effect of a candidate compound on the release of preformed mediators from mast cells (i.e., degranulation) using standard procedures known in the art.

### Screening/Testing for Modulators of PI3KC2β

Any screening technique known in the art can be used to screen for active or positive candidate compounds that modulate PI3KC2β activity/function. The present invention contemplates screens for small molecule modulators, as well as screens for natural proteins or peptides that bind to and modulate PI3KC2β activity or function. For example, natural products or peptide libraries can be screened using assays described herein to identify molecules that have the ability to modulate PI3KC2β activity and/or mast cell activation, e.g., to inhibit mast cell degranulation.

Identification and screening of a molecule is further facilitated by determining structural features of the protein, e.g., using X-ray crystallography, neutron diffraction, nuclear magnetic resonance spectrometry, and other techniques for structural assessment and determination. These techniques provide for the rational design or identification of proteins, peptide fragments, or small molecules that have a modulatory effect on PI3KC2β activity or function.

Another approach uses recombinant bacteriophage to produce large libraries. Using the “phage method” [Scott and Smith, 1990, Science 249:386-390 (1990); Cwirla, et al., Proc. Natl. Acad. Sci., 87:6378-6382 (1990); Devlin et al., Science, 249:404-406 (1990)], very large libraries can be constructed (10⁶-10⁸ chemical entities). A second approach uses primarily chemical methods, of which the Geysen method [Geysen et al., Molecular Immunology 23:709-715 (1986); Geysen et al. J. Immunologic Method 102:259-274 (1987)] and the method of Fodor et al. [Science 251:767-773 (1991)] are examples. Furka et al. [14th International Congress of Biochemistry, Volume 5, Abstract FR:013 (1988); Furka, Int. J. Peptide Protein Res. 37:487-493 (1991)], Houghton [U.S. Pat. No. 4,631,211] and Rutter et al. [U.S. Pat. No. 5,010,175] describe methods to produce a mixture of peptides that can be tested as activators or inhibitors.

Screening phage-displayed random peptide libraries offers a rich source of molecular diversity and represents a powerful means of identifying peptide ligands that bind a receptor molecule of interest (Cwirla, et al., Proc. Natl. Acad. Sci., 87:6378-6382 (1990); Devlin et al., Science, 249:404-406 (1990)). Phage expressing binding peptides are selected by affinity purification with the target of interest. This system allows a large number of phage to be screened at one time. Since each infectious phage encodes the random sequence expressed on its surface, a particular phage, when recovered from an affinity matrix, can be amplified by another round of infection. Thus, selector molecules immobilized on a solid support can be used to select peptides that bind to them. This procedure reveals a number of peptides that bind to the selector and that often display a common consensus amino acid sequence. Biological amplification of selected library members and sequencing allows the determination of the primary structure of the peptide(s).

Peptides are expressed on the tip of the filamentous phage M13, as a fusion protein with the phage surface protein pilus (at the N-terminus). Typically, a filamentous phage carries on its surface 3 to 5 copies of pili and therefore of the peptide. In such a system, no structural constraints are imposed on the N-terminus; the peptide is therefore free to adopt many different conformations, allowing for enhanced diversity.

In another aspect, synthetic libraries [Needels et al., Proc. Natl. Acad. Sci. USA 90:10700-4 (1993); Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety], and the like can be used in screening assays. The LOPAC Library of Pharmacologically Active Compounds for Assay Validation and High Throughput Screening (Sigma, Catalog #SC001) may also be used in screening assays. In addition, a number of chemical libraries are available for screening from the Development Therapeutics program at The National Cancer Institute site on the worldwide web via Developmental Therapeutics Program (DTP) National Cancer Institute National Institutes of Health website gov/branches/dscb/div2 explanation.html. These include “NCI Diversity Set #1” and the “Approved Oncology Drugs Set.”

Alternatively, or in addition, the effect of a candidate compound may be tested in screens using immune cells, such as mast cells obtained from tissues including skin and lung or differentiated from stem cells isolated from blood or bone marrow. In addition, several mast cell lines exist, such as the MC-9 or HC-1 cell lines (Demo et al. 1999. Cytometry 36:340-348; Galli et al. 1982. J Cell Biol 95:435-444; Moon et al. 2011. European Journal of Pharmacology 671:128-132; Galli et al. J Cell Biol 1982; 95:435-444; Demo et al. Cytometry 1999; 36:340-348). For example, one may assess the effects of the candidate compound on FCεR1 receptor oligomerization and activation and resultant release of preformed mediators, such as histamine, proteases, and cytokines stored in cytoplasmic granules. A positive candidate, if it were an inhibitor, e.g., would reduce the amount of histamine, proteases, and cytokines released from cytoplasmic granules in mast cells.

The methods used to measure the effect of the candidate compound on mast cells, more particularly, on the release of preformed mediators, may include standard procedures known to those skilled in the art. The release of preformed mediators from mast cells can, for example, be measured by harvesting cellular supernatants and assaying same by immunoblotting or on the basis of biological activity present therein. The level of expression of a gene or gene product (protein) may furthermore be determined by a method selected from, but not limited to, cDNA microarray, reverse transcription-polymerase chain reaction (RT-PCR), real time PCR and proteomics analysis. Other means such as electrophoretic gel analysis, enzyme immunoassays (ELISA assays), Western blots, dot blot analysis, Northern blot analysis and in situ hybridization may also be contemplated for use, although it is to be understood that the former assays that are noted (eg. micrarrays, RT-PCR, real time PCR and proteomics analysis) provide a more sensitive, quantitative and reliable measurement of genes or gene products that are modulated by a candidate compound. Sequences of the genes or cDNA from which probes are made (if needed) for analysis may be obtained, e.g., from GenBank.

### In Vitro/In Vivo Methods

As described herein, the present invention is directed to a method for screening to identify agents that inhibit PI3KC2β, the method comprising the steps of: contacting PI3KC2β or functional fragment thereof with at least one agent of a library of candidate agents/compounds to determine if the at least one agent inhibits kinase activity of the PI3KC2β or functional fragment thereof. Exemplary nucleic and amino acid sequences of PI3KC2β or a functional fragment thereof are presented in SEQ ID NOs: 1-4. In a particular embodiment thereof, an agent may bind and inhibit PI3KC2β activity.

Kinase assays may be performed in 10 mM Tris, 100 mM NaCl, 1 μM ATP, 5 nM phosphatidylinositol in the presence of 10 nM of each library compound. The final kinase reaction volume is 50 μl. Following a 30 minute kinase reaction, 50 μl of the luminescent buffer reagent will be added to each well and allowed to incubate for 10 minutes prior to reading luminescence.

Kinase assays for modulators of other class II PI3 kinases have been described in, for example, U.S. Pat. Nos. 6,436,671 and 6,700,467. Methods for performing same are described therein and are incorporated herein in their entirety.

In one embodiment, screens for PI3KC2β inhibitors are performed using a high-throughput assay with one of several chemical libraries that are available. Active wild type PI3KC2β and a kinase dead point mutant will be generated in baculovirus using methods known in the art (Sinnamon et al. 2010. Protein expression and purification 73:167-176). PI3KC2β kinase activity can be assessed using a variety of assay systems. An exemplary assay system is the ADP-Glo Kinase Assay System (Promega). In accordance with same, PI3KC2β kinase activity can be assessed by co-incubating PI3KC2β with its substrate phosphatidylinositol (PI). This assay is extremely sensitive, has a wide dynamic range, is amenable to high-throughput screening, and does not require radiolabelled nucleotides to detect kinase activity. The ADP-Glo Kinase Assay System detects the generation of ADP from ATP mediated by the phosphorylation of PI by PI3KC2β to generate PI3P. A compound that inhibits PI3KC2β's kinase activity will result in the generation of less ADP, which would then be detected by the assay. Positive hits will be secondarily screened against the class I PI3K, p110α, as well as several other kinases including the epidermal growth factor receptor kinase, ERK map kinase, and Janus Kinase 3 to determine specificity. Those compounds with the highest specificity and sensitivity will be crystallized as described below to identify the mechanism of inhibition as well as to develop strategies to generate more specific high affinity chemical inhibitors of PI3KC2β's kinase activity.

The composition of the kinase assay system is described, for example, in U.S. Pat. No. 7,770,310, the entire content of which is incorporated herein in its entirety. In a particular embodiment thereof, a buffered detergent solution having a pH in the range of about pH 6.0 to about pH 8.0 is provided and utilized that comprises DTAB whose concentration in the reagent composition is in the range of about 0.05% to about 2% (w/v) and optionally comprises NaF whose concentration in the reagent composition is in the range of about 1 mM to about 20 mM and optionally comprises THESIT whose concentration in the reagent composition is in the range of about 1% to about 5%. Lyophilized luciferase, preferably a luciferase with the sequence of SEQ ID NOs: 1, 2, 3, or 4, most preferably SEQ ID NOs: 2 or 4 of U.S. Pat. No. 7,770,310 may also be utilized. SEQ ID NOs: 1-4 of U.S. Pat. No. 7,770,310 are incorporated herein by reference in their entirety. When combined with the buffered detergent solution to create the reagent composition, it is preferable for the luciferase to be at a concentration of 1 μg/ml or greater, and more preferably at a concentration of 80 μg/ml or greater. The container comprising lyophilized luciferase preferably further comprises lyophilized luciferin.

Co-crystallization of PI3KC2β with compounds identified in screening methods described herein can be performed as follows. The catalytic subunit of PI3KC2β will be purified from baculovirus-infected SD insect cells and purified to homogeneity. The protein will be concentrated to between 5 and 10 mg/ml and incubated with compounds identified above at a molar ratio of 5-10 compound:protein. Crystallization trials will be set up using commercially available and home-made screening kits and a TTPLabTech Mosquito crystallization robot. Crystals will be analyzed, and data collected, on a Rigaku MicroMax-007 rotating anode with Raxis IV++ image plate detector.

Computer assisted three dimensional reconstruction of PI3KC2β may also be used to identify and design inhibitors or activators of PI3KC2β that modulate PI3KC2β activity or to provide guidance on which basis a more targeted screen can be designed.

As described elsewhere herein, an important aspect of the present screening methods is to assess agents identified in primary screens with respect to their ability to bind to and/or inhibit Class I or other Class II PI3 kinases. In that it is an objective of the present screening methods to identify inhibitors that are specific for Class II PI3 kinases and, more particularly, are specific for PI3KC2β, such secondary or tertiary screens to evaluate specificity are envisioned herein.

Further to the above, once identified, active candidate agents (e.g., PI3KC2β inhibitors) are assessed in secondary screens that may, for example, be cell-based assays. Cells useful for such assays include, without limitation, those cells described herein, including BMMCs, and various mast cell lines which are isolated or derived from a mammal. Mast cells for use in the secondary assays can be isolated or derived from, for example, humans, other primates, mice, and rats. Secondary screens may also be performed in vivo, using animal model systems such as those described herein and known in the art. Such animal model systems include those which recapitulate aspects of human IgE-mediated allergic disorders, including allergic rhinitis, asthma, anaphylaxis, or immediate type hypersensitivity reactions.

As described herein, a secondary cell-based assay may be performed essentially as a method for inhibiting mast cell activation, the method comprising the steps of: contacting a population of mast cells with either an active candidate agent (e.g., a PI3KC2β inhibitor identified in a primary screen) or a control substance and evaluating the ability of the active candidate agent relative to that of the control substance to reduce or inhibit mast cell activation, wherein if the active candidate agent reduces or inhibits mast cell activation relative to the control substance, the active candidate agent is identified as an inhibitor of mast cell activation and can be confirmed as a bona fide PI3KC2β inhibitor in a cellular context. As described herein, secondary cell-based assays can be performed in cell culture (in vitro) or in the context of an animal (in vivo). As described herein, an active candidate agent may be identified by analyses based on computer modeling of three dimensional structure and/or by primary screens of libraries.

As taught herein, in vitro mast cell activation can be evaluated or measured by detecting an increase in release of preformed mediators, such as histamine, proteases, beta-hexosaminadase and cytokines stored in cytoplasmic granules from the mast cell. In addition, FcεRI stimulated activation of KCa3.1 channel activity and calcium influx can be assessed. Typically, mast cell activation resulting in degranulation (i.e., release of preformed mediators) is triggered by FCεR1 receptor oligomerization and activation. Activation of mast cells following stimulation with phorbol myristate acetate and ionomycin can also be assessed. Accordingly, secondary cell-based assays call for contacting the population of mast cells in the presence of a mast cell activator or activators that are added before, concurrently, or after contacting with the active candidate agent. In a particular embodiment, the population of mast cells is pre-treated with the active candidate agent and then incubated in the presence of a mast cell activator or activators. In vivo, activation is an ongoing process, so an active candidate agent can be administered before, concurrently, or after exposure to a mast cell activator or activators. Administration concurrently or after exposure to a mast cell activator or activators will stop the recruitment of new mast cells from getting activated. Experimental protocols that can be used to measure mast cell activation and reduction or inhibition thereof are described in detail herein and are understood in the art.

### Candidate Compounds and Agents

As used herein, an “agent”, “candidate compound”, or “test compound” may be used to refer to, for example, nucleic acids (e.g., DNA and RNA), carbohydrates, lipids, proteins, peptides, peptidomimetics, small molecules and other drugs. An agent may also refer to short hairpin RNA (shRNA), small interfering RNA (siRNA), and neutralizing and/or blocking antibodies.

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. In a particular embodiment, shRNA or siRNA is designed and used to inhibit PI3KC2β.

Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene.

As described herein, an agent identified using the method of the present invention that is a “modulator of mast cell activation” is defined as an agent that is capable of modulating (e.g., increasing or decreasing) activation of mast cells. Such an agent may be identified by its ability to effect a change in FCεR1 receptor oligomerization and activation and/or release of preformed mediators, such as histamine, proteases, and cytokines stored in cytoplasmic granules of the mast cell. As detailed below, experimental protocols of utility in evaluating the above indicators of mast cell activation are described in detail herein and are understood in the art. Such experimental protocols, include, but are not limited to, measuring Ca⁺² influx, KCa3.1 channel activity, mast cell degranulation (using, e.g., β-hexoasamidase), and susceptibility to passive systemic anaphylaxis.

As taught herein, the change effected by an agent that is a modulator of PI3KC2β activity or mast cell activation is determined relative to that of a population of mast cells incubated in parallel in the absence of the agent or in the presence of a control agent (as described below), either of which is analogous to a negative control condition.

The term “control substance”, “control agent”, or “control compound” as used herein refers a molecule that is inert or has no activity relating to an ability to modulate a biological activity. With respect to the present invention, such control substances are inert with respect to an ability to modulate PI3KC2β activity or mast cell activation. Exemplary controls include, but are not limited to, solutions comprising physiological salt concentrations.

It is to be understood that agents capable of modulating PI3KC2β activity or mast cell activation, as determined using in vitro methods described herein, are likely to exhibit similar modulatory capacity in applications in vivo.

Modulatory agents identified using the screening methods of the present invention and compositions thereof can thus be administered for therapeutic treatments. In therapeutic applications, modulatory agents that inhibit PI3KC2β activity/mast cell activation and compositions thereof are administered to a patient susceptible to or suffering from an IgE-dependent allergic disorder in an amount sufficient to at least partially arrest a symptom or symptoms of the disease and its complications. An amount adequate to accomplish this is defined as a “therapeutically effective amount or dose.” Amounts effective for this use will depend on the severity of the disease and the weight and general state of the patient.

Examples of IgE-dependent allergic disorders that may be treated using inhibitors of PI3KC2β activity/mast cell activation include, without limitation, allergic rhinitis, asthma, anaphylaxis, and immediate type hypersensitivity.

The basic molecular biology techniques used to practice the methods of the invention are well known in the art, and are described for example in Sambrook et al., 1989, Molecular Cloning: A Laboratory Manual, Cold Spring Harbor Laboratory, New York; Ausubel et al., 1988, Current Protocols in Molecular Biology, John Wiley & Sons, New York; and Ausubel et al., 2002, Short Protocols in Molecular Biology, John Wiley & Sons, New York). Agents Identified by the Screening Methods of the Invention

The invention provides methods for identifying agents (e.g., candidate compounds or test compounds) that modulate (inhibit or promote) PI3KC2β activity/mast cell activation. Agents that are capable of inhibiting PI3KC2β activity/mast cell activation, as identified by the screening method of the invention, are useful as candidate therapeutics for IgE-mediated allergic disorders. This assertion is based on results presented herein that demonstrate for the first time that PI3KC2β is essential for FcεR1 activation of KCa3.1 and Ca²⁺ influx in mast cells, and that TRIM27 plays a role in negatively regulating KCa3.1 channel activity and FcεR1 activation of mast cells in vitro and in vivo at least in part via inhibiting PI3KC2β activity.

A list of IgE-mediated allergic disorders that may be treated using an agent identified using a method of the invention includes, without limitation: allergic rhinitis, asthma, anaphylaxis, and immediate type hypersensitivity.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

### Therapeutic Uses of Agents Identified

The invention provides for treatment of IgE-mediated allergic disorders by administration of a therapeutic agent identified using the above-described methods. Such agents include, but are not limited to proteins, peptides, protein or peptide derivatives or analogs, antibodies, nucleic acids, and small molecules.

The invention provides methods for treating patients afflicted with an IgE-mediated allergic disorder comprising administering to a subject an effective amount of a compound identified by the method of the invention. In a particular aspect, the compound is substantially purified (e.g., substantially free from substances that limit its effect or produce undesired side-effects). The subject is particularly an animal, including but not limited to animals such as cows, pigs, horses, chickens, cats, dogs, etc., and is more particularly a mammal, and most particularly a human. In a particular embodiment, a non-human mammal or a human is the subject.

Formulations and methods of administration that can be employed when the compound comprises a nucleic acid are described herein and known in the art; additional appropriate formulations and routes of administration are described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, e.g., an inflammatory site, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

### Pharmaceutical Compositions

The present invention also provides pharmaceutical compositions. Such compositions comprise a therapeutically effective amount of an agent and a pharmaceutically acceptable carrier. In a particular embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of an IgE-mediated allergic disorder (e.g., allergic rhinitis, asthma, anaphylaxis, and immediate type hypersensitivity reactions) can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

### Nucleic Acids

The invention provides methods of identifying agents capable of modulating PI3KC2β activity/mast cell activation. Accordingly, the invention encompasses administration of a nucleic acid encoding a peptide or protein capable of modulating PI3KC2β activity/mast cell activation, as well as antisense sequences or catalytic RNAs capable of interfering with PI3KC2β activity/mast cell activation. Exemplary nucleic acid sequences of PI3KC2β or a functional fragment thereof are presented in SEQ ID NOs: 1 and 3, which encode SEQ ID NOs: 2 and 4, respectively. Also encompassed herein are nucleic and amino acid sequences (SEQ ID NOs: 9 and 10) of human tripartite motif containing 27 (TRIM27), which sequences may also be useful in the screening methods described herein. See also NCBI Reference Sequences NM_{—}006510.4 and NP_{—}006501.1, respectively, the entire content of each of which is incorporated herein by reference.

Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein capable of modulating PI3KC2β activity/mast cell activation, such nucleic acid being part of an expression vector that expresses the peptide or protein in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding a desired polypeptide to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a particular embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a DNA coding for a peptide or protein capable of modulating PI3KC2β activity/mast cell activation may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

Unless defined otherwise, all technical and scientific terms used herein have the same meaning as commonly understood by one of ordinary skill in the art to which this invention belongs.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting, since the scope of the present invention will be limited only the appended claims.

### Example I

### Materials and Methods

Cells and Constucts.

Bone marrow derived mast cells (BMMC) were generated from 6-8 week old TRIM27^{+/+} and TRIM27^{-/-} mice that were backcrossed 8 generations to C57Bl/6 mice as previously described (27). TRIM27^{-/-} mice were generated from the ES cell line 345D11 (The Center for Disease Modeling at The University of Toronto), which contained the exon-trapping plasmid pUPA located between exon 1 and 2 of TRIM27 on mouse chromosome 13 and has been previously described (Cai et al. 2011. Proc Natl Acad Sci USA 108:20072-20077). Bone marrow cells were cultured for 6-8 weeks in RPMI supplemented with IL-3 (20 ng/ml), stem cell factor (100 ng/ml), and 10% FCS. Generation of a pure population of mast cells after 6 weeks of culture was verified by staining with PE-labeled anti-FcεR1 antibody followed by FACS analysis.

Antibodies:

Anti-TRIM27 antibodies were purchased from IBL America. Anti-PI3KC2β antibody 3E2 (Novus Biologicals, Littleton, Co) was used to immunoblot mouse PI3KC2β.

Whole Cell Patch Clamp:

Whole cell patch clamping was performed on TRIM27^{+/+} and TRIM27^{-/-} BMMCs that were first sensitized overnight with anti-DNP IgE and then stimulated with DNP-HSA using conditions previously described (5). Briefly, the standard pipette solution contained 140 mM KCl, 2 mM MgCl₂, 10 mM Hepes, 2 mM Na⁺-ATP and 0.1 mM GTP, pH 7.3. The standard external solution contained 140 mM NaCl, 5 mM KCl, 2 mM CaCl₂, 1 mM MgCl₂ and 10 mM Hepes, pH 7.3. Whole-cell currents were recorded using an Axoclamp 200 B amplifier (Axon Instruments, Foster City, Calif., USA), and currents were evoked by applying voltage commands to a range of potentials in 10 mV steps from a holding potential of 20 mV. For measuring the membrane potential, ruptured patch was used in current clamp mode as described in (34).

To verify that PI3KC2β mediated activation of KCa3.1 via the generation of PI3P, PI3P (100 nM) was added into the pipette solution during patch clamping in BMMCs in which PI3KC2β was knocked down using siRNA (32). PI(3)P diC16 [C₄₁H₄₅Na₃O₁₆P₂ (C₆)] was purchased from Echelon Biosciences and used according to specifications at a concentration of 100 nM in the pipette solution.

Intracellular Ca2⁺ Activity:

BMMCs from TRIM27^{+/+} and TRIM27^{-/-} mice were sensitized overnight with anti-DNP IgE (100 ng/ml) and subsequently loaded with 5 μM Fura-2 AM ester (Molecular Probes) in RPMI medium for 30 min at room temperature, washed and then resuspended in RPMI. Cells were attached to poly(L)lysine-coated coverslips for 20 min in a RC-20 bath flow chamber (Warner Instrument Corp., Hamden, Conn.) and fura-2 fluorescence was recorded (Delta Ram; PTI Inc., South Brunswick, N.J.) at excitation wavelengths of 340 and 380 nm. Data are represented as the ratio 340/380 after background subtraction. Intracellular Ca²⁺ was measured before and after the perfusion of DNP-HSA in the HBSS buffer in the presence of 1 mM extracellular Ca²⁺.

β-Hexosaminadase Release and Cytokine Production:

BMMCs were plated at 1×10⁶ cells/96 well plate in media supplemented with DNP-IgE antibody for 4 hours. Cells were then washed and stimulated with various concentrations of DNP-HSA for 30 minutes in Tyrode's buffer (10 mM HEPES, pH 7.4, 130 mM NaCl, 5 mM KCl, 1.4 mM CaCl₂, 1 mM MgCl₂, 5.6 mM glucose and 0.1% (wt/vol) BSA. Cells were then spun at 1200 RPMs and β-hexosaminadase was measured in the supernatant by incubating 30 μl of supernatant with 3.3 ul of p-nitrophenyl-N-acetyl-β-D-glucosamide (10 mM) diluted in 0.2 M citrate buffer, ph 4.5 for 1.5 hours at 37° C. The reaction was then stopped by adding 135 μl of a 0.1 M Na₂CO₃/0.1 M NaHCO₃ solution and then assayed on an ELISA plate reader at an OD @ 405 nm. β-hexosaminadase was measured in the pellet following a similar protocol with the exception that the cell pellet was lysed in Tyrode's buffer with 1% triton.

To assay for cyokines, mast cells were stimulated as above, total RNA was isolated using Trizol reagent and then reverse transcribed using random hexamer primers. Quantitative PCR was then assessed using SYBR Green 1 by iCycler iQ (Biorad) using cytokine specific primers purchased from Qiagen.

siRNA Knockdown of PI3KC2β:

BMMCs were transfected with 2 independent siRNAs to PI3KC2β using lipofectamine RNAiMAX reagent and cells were studied 48 hours after transfection. Silencing of PI3KC2β was confirmed by RT PCR and immunoblotting. The siRNAs used were: siRNA 1, 5′-CCAAGAUCUCUCAGCCUAATT-3′ (SEQ ID NO: 5; sense sequence) and 5′-UUAGGCUGAGAGAUCUUGGAG-3′ (SEQ ID NO: 6; antisense sequence); siRNA 2, 5′GGGUGGUCCAGUCUGUCAATT-3′ (SEQ ID NO: 7; sense sequence) and 5′-UUGACAGACUGGACCACCCTG-3′ (SEQ ID NO: 8; antisense sequence).

Passive Systemic Anaphylaxis:

TRIM27^{+/+} and TRIM27^{-/-} mice were first sensitized with anti-DNP IgE (30 μg) administered by intraperitoneal injection. After 5 hrs, mice were challenged with either DNP-HSA (50 μg) or PBS control and body temperature was measured before and then at 5 minute intervals following challenge using a rectal probe (38). Blood was also collected 30 minutes following challenge and assayed for histamine as described (1).

Immunoblot:

BMMCs from TRIM27^{+/+} and TRIM27^{-/-} mice were senstitized for 4 hours with anti-DNP IgE (100 ng/ml) and then stimulated with DNP-HSA for various periods of time. Lysates were then immunoblotted with various antibodies as previously described (28, 32)

### Results

PI3KC2β Activation is Required for FcεR1 Stimulated KCa3.1 Channel Activity and Ca2+ Influx in BMMCs.

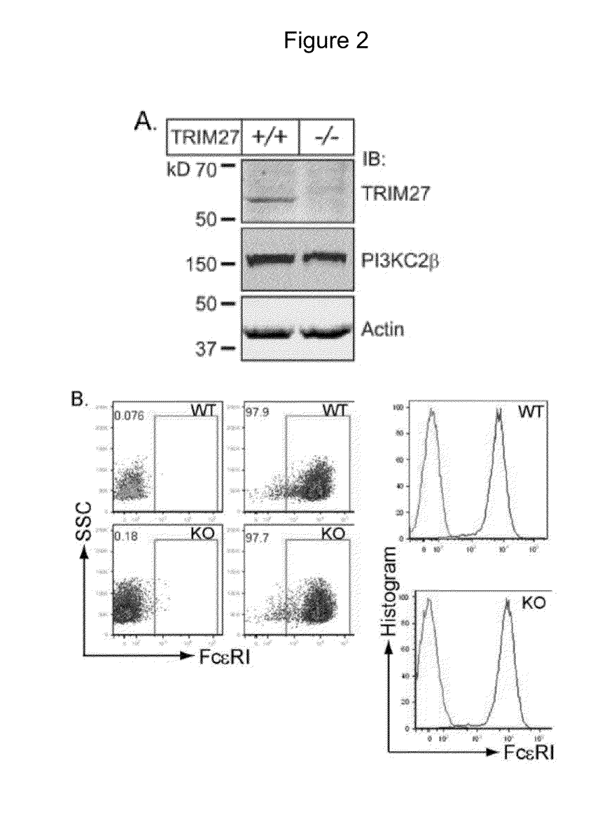

Previous studies have shown that activation of KCa3.1 in CD4 T cells is mediated via TCR stimulated activation of PI3KC2β, which functions to generate the pool of PI3P required for KCa3.1 channel activation (29). To test whether PI3KC2β is also required for KCa3.1 activation by FcεR1, BMMCs were generated from TRIM27^{+/+} and TRIM27^{-/-} mice (FIGS. 1A and B). As shown herein, FcεR1 stimulated KCa3.1 activation was decreased in TRIM27^{+/+} BMMCs following siRNA knockdown of PI3KC2β with 2 independent siRNAs to PI3KC2β (FIG. 2A-C). This was due to decreased PI3P because dialyzing siRNA knockdown cells with PI3P restored FcεR1 stimulated KCa3.1 channel activity (FIGS. 2B and C), while other phosphoinositides such as PI4P and PI(3,4,5)P₃ failed to rescue (FIG. 2C). The decrease in KCa3.1 channel activity also led to decreased FcεR stimulated Ca²⁺ influx; both the acute rise as well as the sustained plateau phase of Ca²⁺ influx was decreased in PI3KC2β knocked down cells (FIG. 2D).

FcεR1 Stimulated KCa3.1 Channel Activity and Ca2+ Influx is Increased in TRIM27−/−Mast Cells.

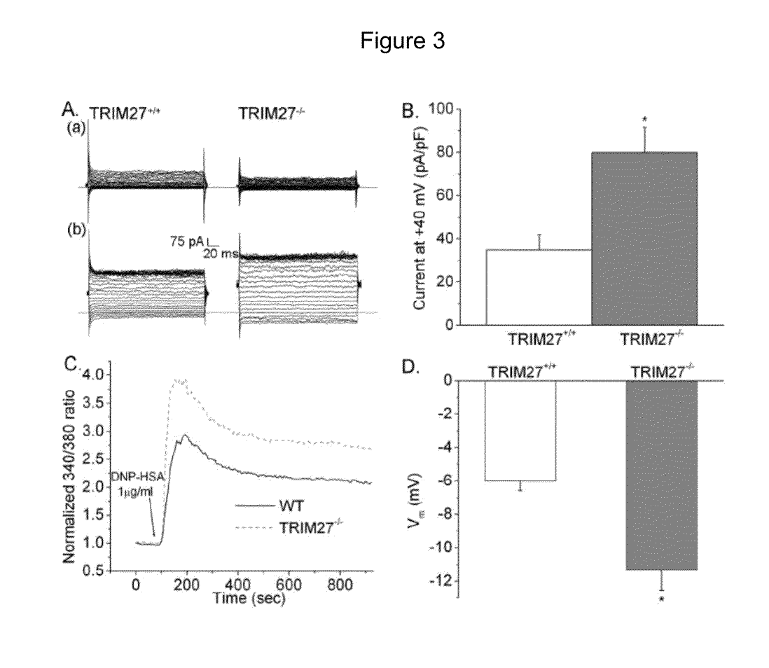

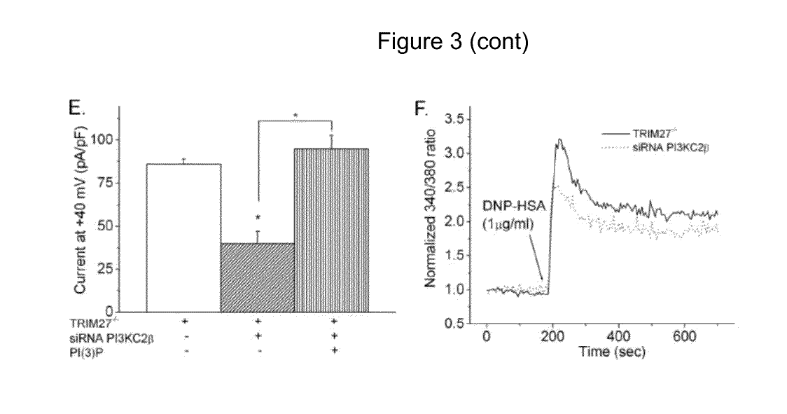

The finding that PI3KC2β is required for FcεR1 stimulated KCa3.1 channel activity and Ca²⁺ influx suggested that TRIM27, via inhibition of PI3KC2β, may also function to negatively regulate mast cells. BMMCs were generated from TRIM27^{+/+} and TRIM27^{-/-} mice. TRIM27^{-/-} BMMCs differentiated normally and expressed similar levels of FcεR as TRIM27^{+/+} cells (FIG. 1B). While basal KCa3.1 channel activity was similar between TRIM27^{+/+} and TRIM27^{-/-} BMMCs, FcεR1 stimulated KCa3.1 channel activity was increased about 50% in TRIM27^{-/-} BMMCs (FIGS. 3A and B). The increase in KCa3.1 channel activity was likely due to increased activity of PI3KC2β as siRNA knockdown of PI3KC2β in TRIM27^{-/-} BMMCs decreased Ka3.1 channel activity and Ca²⁺ flux to basal levels (FIGS. 3C and D).

Consistent with the increase in KCa3.1 channel activity, TRIM27^{-/-} BMMCs also had an increase in FcεR1 stimulated Ca²⁺ influx (FIG. 3E) as well as a more negative membrane potential (FIG. 3F). Both the acute rise and the sustained plateau phase of Ca²⁺ influx was increased in TRIM27^{-/-} BMMCs. The increased Ca²⁺ influx was likely due to changes in membrane potential; TRIM27^{-/-} mast cells had a more negative membrane potential, which would provide the driving force for increased Ca²⁺ influx.

FcεR1 Stimulated 13-Hexoasamidase Release and Cytokine Production is Increased in TRIM27^{-/-} BMMCs.

FcεR1 mediated rapid degranulation and cytokine production is dependent upon Ca²⁺ entry into mast cells (2, 14). To assess whether increased Ca²⁺ influx leads to increased degranulation of TRIM27^{-/-} BMMCs, FcεR1 stimulated β-hexoasamidase release was assessed. β-hexoasamidase is stored in preformed granules in mast cells and is released into the supernatant following FcεR1 stimulation. Without stimulation, the amount offl-hexoasamidase released was similar between TRIM27^{+/+} and TRIM27^{-/-} BMMCs (FIG. 4A). However, after stimulation a significant increase in β-hexoasamidase release was seen in TRIM27^{-/-} BMMCs (FIG. 4A). In addition, FcεR1 stimulated induction of mRNA for the cytokines TNFα, IL-6, and IL-13 was increased in TRIM27^{-/-} BMMCs (FIG. 4B i, ii and iii). Thus, increased FcεR1 stimulated Ca²⁺ influx in TRIM27^{-/-} BMMCs is associated with increased degranulation and production of inflammatory cytokines that mediate allergic responses.

TRIM27^{-/-} Mice are More Susceptible to Passive Systemic Anaphylaxis.

To assess whether changes in BMMCs in vitro are also relevant in vivo, TRIM27^{-/-} and TRIM27^{+/+} mice were sensitized intraperitoneally (IP) with anti-DNP IgE. After resting overnight, mice were then challenged IP with DNP-HAS or saline control and body temperature and serum histamine levels were assessed over time. The decrease in body temperature following treatment with antigen was significantly increased in TRIM27^{-/-} mice as well as the increase in serum histamine levels 30 minutes after challenge (FIG. 5). Thus, these findings indicate that mast cells in vivo in TRIM27^{-/-} mice are more sensitive to degranulation following antigen stimulation.

Activation of Proximal and MAP Kinase Signaling Pathways are Similar in TRIM27^{+/+} and TRIM27^{-/-} BMMCs.

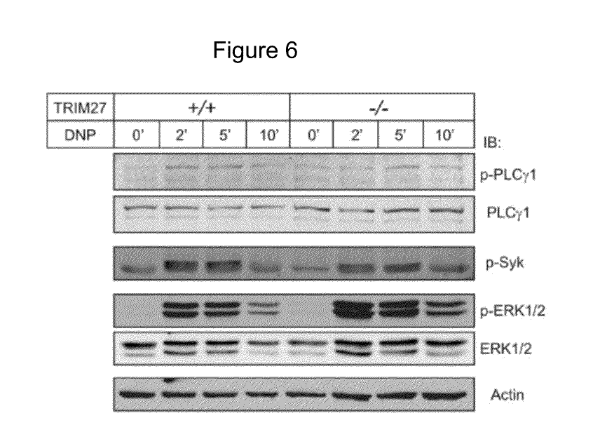

If TRIM27 mediates the inhibition FcεR1 stimulated KCa3.1 activation via inhibition of PI3KC2β, it would be reasonable to predict that tyrosine phosphorylation of proximal signaling molecules such as PLCγ1 and Syk should be similar between TRIM27^{+/+} and TRIM27^{-/-} BMMCs. TRIM27^{-/-} and TRIM27^{+/+} BMMCs were stimulated with DNP-HSA for various times following sensitization with DNP-IgE, and activation of signaling molecules was assessed by western blotting with antiphospho-specific antibodies. These studies demonstrated tyrosine phosphorylation of PLCγ1 and Syk was similar between TRIM27^{+/+} and TRIM27^{-/-} BMMCs (FIG. 6). In addition, activation of ERK MAP kinase was also similar between TRIM27^{-/-} and TRIM27^{+/+} cells (FIG. 6).

### Discussion

IgE stimulated influx of extracellular Ca²⁺ via CRAC channels in mast cells is critical for FcεR1 stimulated degranulation and cytokine production (1, 35). CRAC channel mediated influx of Ca²⁺ is also regulated by other channels that include KCa3.1 and TRPM4, which by regulating membrane potential play critical roles in modulating IgE stimulated Ca²⁺ influx (27, 34). Thus, understanding the mechanisms whereby KCa3.1 and TRPM4 are regulated in mast cells will likely uncover important regulators of allergic responses and provide new therapeutic targets to treat allergic disease. Further to this objective, the present inventors demonstrate for the first time herein that both PI3KC2β and TRIM27 play critical but opposite roles in FcεR1 stimulated activation of KCa3.1, Ca²⁺ influx, degranulation, and cytokine production in BMMCs. These findings are, moreover, relevant and applicable to in vivo circumstances, since IgE-mediated anaphylactic response is increased in TRIM27−/− mice.

KCa3.1 is an intermediate-conductance Ca^{2±}-activated K+ channel. By mediating the efflux of K⁺, KCa3.1 functions to maintain a negative membrane potential, which provides the electrical force to drive Ca²⁺ entry into mast cells, and some subsets of CD4 T cells and B lymphocytes (3, 10, 37). It has been known for some time that binding of Ca²⁺ to calmodulin bound to the carboxy-terminus (CT) of KCa3.1 is critical for KCa3.1 activation (7, 13, 20, 26). More recently, studies in CD4 T cells have identified a second signaling pathway that is required for KCa3.1 activation. These studies demonstrated that following TCR activation, PI3KC2β is recruited to the immunological synapse leading to the generation of PI3P, which is required for NDPKB to phosphorylate Histidine 358 in the CT of KCa3.1, thereby providing the second signal for KCa3.1 channel activation (29, 31). Results presented herein demonstrate that PI3KC2β is also required for activation of KCa3.1 in BMMCs and, via KCa3.1 activation, is required for FcεR1 stimulated Ca²⁺ influx and degranulation. This is supported by the instant finding that siRNA knockdown of PI3KC2β inhibits FcεR1 stimulated KCa3.1 channel activity and Ca²⁺ influx. Moreover, this inhibition is due to decreased levels of PI3P because dialyzing PI3KC2β siRNA transfected cells with PI3P, but not other phosphoinositides, during whole cell patch clamp rescued KCa3.1 channel activity. Thus, these findings when taken together demonstrate that PI3KC2β functions downstream of FcεR1, and suggests that PI3KC2β may be a common mechanism for linking other antigen receptors, such the B cell antigen receptor, to KCa3.1 activation.

TRIM family members have been shown to regulate a plethora of biological responses including innate and adaptive response to infection, cell proliferation, anti-viral responses, and development by functioning as a novel family of E3 ligases (21-23). The present inventors recently found that TRIM27 downregulates TCR stimulated activation of KCa3.1 and Ca²⁺ influx in CD4 T cells by ubiquitinating and inhibiting PI3KC2β enzyme activity (4). The present results reveal that TRIM27 also functions as a negative regulator of mast cells. BMMCs derived from TRIM27^{-/-} mice exhibit increased FcεR1 stimulated KCa3.1 channel activity, Ca²⁺ influx, degranulation, and production of inflammatory cytokines when compared with TRIM27^{+/+} BMMCs. Moreover, evidence is presented in knockout mice that TRIM27 functions to regulate IgE-mediated degranulation of mast cells and anaphylactic response in vivo. Consistent with TRIM27 mediating its effects via increased KCa3.1 channel activity, TRIM27^{-/-} BMMCs had a more negative membrane potential following FcεR1 stimulation, which would then provide a more favorable electrochemical driving force for Ca²⁺ influx. In addition, differences in tyrosine phosphorylation of proximal signaling pathways downstream of FcεR1 stimulation, such as phospholipase Cγ1 and Syk, were not detected in TRIM27^{-/-} BMMCs as would be predicted if TRIM27 mediated its effects predominantly via the regulation of PI3KC2β and KCa3.1.

In comparison with the better studied class I PI3Ks, much less is known about class II PI3Ks (6, 33). Nevertheless, studies over the past several years have demonstrated critical roles for the class II PI3Ks in a number of biological processes (17, 18, 29). The distinct biological roles for class II PI3Ks are likely mediated by their ability to generate a different lipid product in vivo, PI3P, which activates different intracellular signaling pathways than the class I PI3Ks, which generate predominately PI(3,4,5)P₃ and PI(4,5)₂ (6, 33). This model is consistent with our findings that PI3P is the only phosphotidylinositol generated downstream of PI3KC2β that is required for KCa3.1 activation (29, 32). Moreover, results reported herein that PI3KC2β plays a critical role in FCεR and mast cell activation suggests that identification of a specific pharmacological inhibitor of PI3KC2β provides a unique opportunity to more surgically treat allergic disease with such an agent. Specific pharmacological inhibitors of PI3KC2β would have a better safety profile than drugs being developed to target the better studied class I PI3Ks since PI3KC2β^{-/-} mice do not display overt abnormalities (12).

### REFERENCES

1. Baba, Y., K. Nishida, Y. Fujii, T. Hirano, M. Hikida, and T. Kurosaki. 2008. Essential function for the calcium sensor STIM1 in mast cell activation and anaphylactic responses. Nat Immunol 9:81-88.
2. Bischoff, S. C. 2007. Role of mast cells in allergic and non-allergic immune responses: comparison of human and murine data. Nat Rev Immunol 7:93-104.
3. Cahalan, M. D., H. Wulff, and K. G. Chandy. 2001. Molecular properties and physiological roles of ion channels in the immune system. J Clin Immunol 21:235-252.
4. Cai, X., S. Srivastava, Y. Sun, Z. Li, H. Wu, L. Zuvela-Jelaska, J. Li, R. S. Salamon, J. M. Backer, and E. Y. Skolnik. 2011. TRIM27 negatively regulates CD4 T cell activation via the ubiquitination and inhibition of PI3KC2β. Proceedings of the National Academy of Sciences: [In Press].
5. Duffy, S. M., G. Cruse, C. E. Brightling, and P. Bradding. 2007. Adenosine closes the K+ channel KCa3.1 in human lung mast cells and inhibits their migration via the adenosine A2A receptor. Eur J Immunol 37:1653-1662.
6. Falasca, M., W. E. Hughes, V. Dominguez, G. Sala, F. Fostira, M. Q. Fang, R. Cazzolli, P. R. Shepherd, D. E. James, and T. Maffucci. 2007. The role of phosphoinositide 3-kinase C2alpha in insulin signaling. J Biol Chem 282:28226-28236.
7. Fanger, C. M., S. Ghanshani, N. J. Logsdon, H. Rauer, K. Kalman, J. Zhou, K. Beckingham, K. G. Chandy, M. D. Cahalan, and J. Aiyar. 1999. Calmodulin mediates calcium-dependent activation of the intermediate conductance KCa channel, IKCa1. J Biol Chem 274:5746-5754.
8. Feske, S., Y. Gwack, M. Prakriya, S. Srikanth, S. H. Puppel, B. Tanasa, P. G. Hogan, R. S. Lewis, M. Daly, and A. Rao. 2006. A mutation in Orail causes immune deficiency by abrogating CRAC channel function. Nature 441:179-185.
9. Galli, S. J., J. Kalesnikoff, M. A. Grimbaldeston, A. M. Piliponsky, C. M. Williams, and M. Tsai. 2005. Mast cells as “tunable” effector and immunoregulatory cells: recent advances. Annu Rev Immunol 23:749-786.
10. Ghanshani, S., H. Wulff, M. J. Miller, H. Rohm, A. Neben, G. A. Gutman, M. D. Cahalan, and K. G. Chandy. 2000. Up-regulation of the IKCa1 potassium channel during T-cell activation. Molecular mechanism and functional consequences. J Biol Chem 275:37137-37149.
11. Gilfillan, A. M., and C. Tkaczyk. 2006. Integrated signalling pathways for mast-cell activation. Nat Rev Immunol 6:218-230.
12. Harada, K., A. B. Truong, T. Cai, and P. A. Khavari. 2005. The class II phosphoinositide 3-kinase C2beta is not essential for epidermal differentiation. Mol Cell Biol 25:11122-11130.
13. Joiner, W. J., R. Khanna, L. C. Schlichter, and L. K. Kaczmarek. 2001. Calmodulin regulates assembly and trafficking of SK4/IK1 Ca2+-activated K+ channels. J Biol Chem 276:37980-37985.
14. Kalesnikoff, J., and S. J. Galli. 2008. New developments in mast cell biology. Nat Immunol 9:1215-1223.
15. Kraft, S., and J. P. Kinet. 2007. New developments in FcepsilonRI regulation, function and inhibition. Nat Rev Immunol 7:365-378.
16. Liou, J., M. L. Kim, W. D. Heo, J. T. Jones, J. W. Myers, J. E. Ferrell, Jr., and T. Meyer. 2005. STIM is a Ca2+ sensor essential for Ca2+-storedepletion-triggered Ca2+ influx. Curr Biol 15:1235-1241.
17. Maffucci, T., F. T. Cooke, F. M. Foster, C. J. Traer, M. J. Fry, and M. Falasca. 2005. Class II phosphoinositide 3-kinase defines a novel signaling pathway in cell migration. J Cell Biol 169:789-799.
18. Maffucci, T., and M. Falasca. 2007. Phosphoinositide 3-kinase-dependent regulation of phospholipase Cgamma. Biochem Soc Trans 35:229-230.
19. Mark Duffy, S., P. Berger, G. Cruse, W. Yang, S. J. Bolton, and P. Bradding. 2004. The K+ channel iKCA1 potentiates Ca2+ influx and degranulation in human lung mast cells. J Allergy Clin Immunol 114:66-72.
20. Maylie, J., C. T. Bond, P. S. Herson, W. S. Lee, and J. P. Adelman. 2004. Small conductance Ca2+-activated K+ channels and calmodulin. J Physiol 554:255-261.
21. Meroni, G., and G. Diez-Roux. 2005. TRIM/RBCC, a novel class of ‘single protein RING finger’ E3 ubiquitin ligases. Bioessays 27:1147-1157.
22. Napolitano, L. M., E. G. Jaffray, R. T. Hay, and G. Meroni. 2011. Functional interactions between ubiquitin E2 enzymes and TRIM proteins. Biochem J 434:309-319.
23. Ozato, K., D. M. Shin, T. H. Chang, and H. C. Morse, 3rd. 2008. TRIM family proteins and their emerging roles in innate immunity. Nat Rev Immunol 8:849-860.
24. Prakriya, M., S. Feske, Y. Gwack, S. Srikanth, A. Rao, and P. G. Hogan. 2006. Orail is an essential pore subunit of the CRAC channel. Nature 443:230-233.
25. Roos, J., P. J. DiGregorio, A. V. Yeromin, K. Ohlsen, M. Lioudyno, S. Zhang, O. Safrina, J. A. Kozak, S. L. Wagner, M. D. Cahalan, G. Velicelebi, and K. A. Stauderman. 2005. STIM1, an essential and conserved component of store-operated Ca2+ channel function. J Cell Biol 169:435-445.
26. Schumacher, M. A., A. F. Rivard, H. P. Bachinger, and J. P. Adelman. 2001. Structure of the gating domain of a Ca2+-activated K+ channel complexed with Ca2+/calmodulin. Nature 410:1120-1124.
27. Shumilina, E., R. S. Lam, F. Wolbing, N. Matzner, I. M. Zemtsova, M. Sobiesiak, H. Mahmud, U. Sausbier, T. Biedermann, P. Ruth, M. Sausbier, and F. Lang. 2008. Blunted IgE-mediated activation of mast cells in mice lacking the Ca2+-activated K+ channel KCa3.1. J Immunol 180:8040-8047.
28. Srivastava, S., P. Choudhury, Z. Li, G. Liu, V. Nadkarni, K. Ko, W. A. Coetzee, and E. Y. Skolnik. 2006. Phosphatidylinositol 3-Phosphate Indirectly Activates KCa3.1 via 14 Amino Acids in the Carboxy Terminus of KCa3.1. Mol Biol Cell 17:146-154.
29. Srivastava, S., L. Di, O. Zhdanova, Z. Li, S. Vardhana, Q. Wan, Y. Yan, R. Varma, J. Backer, H. Wulff, M. L. Dustin, and E. Y. Skolnik. 2009. The class II phosphatidylinositol 3 kinase C2beta is required for the activation of the K+ channel KCa3.1 and CD4 T-cells. Mol Biol Cell 20:3783-3791.
30. Srivastava, S., K. Ko, P. Choudhury, Z. Li, A. K. Johnson, V. Nadkarni, D. Unutmaz, W. A. Coetzee, and E. Y. Skolnik. 2006. Phosphatidylinositol-3 phosphatase myotubularin-related protein 6 negatively regulates CD4 T cells. Mol Cell Biol 26:5595-5602.
31. Srivastava, S., Z. Li, K. Ko, P. Choudhury, M. Albaqumi, A. K. Johnson, Y. Yan, J. M. Backer, D. Unutmaz, W. A. Coetzee, and E. Y. Skolnik. 2006. Histidine phosphorylation of the potassium channel KCa3.1 by nucleoside diphosphate kinase B is required for activation of KCa3.1 and CD4 T cells. Mol Cell 24:665-675.
32. Srivastava, S., Z. Li, L. Lin, G. Liu, K. Ko, W. A. Coetzee, and E. Y. Skolnik. 2005. The phosphatidylinositol 3-phosphate phosphatase myotubularinrelated protein 6 (MTMR6) is a negative regulator of the Ca2+-activated K+ channel KCa3.1. Mol Cell Biol 25:3630-3638.
33. Vanhaesebroeck, B., J. Guillermet-Guibert, M. Graupera, and B. Bilanges. 2010. The emerging mechanisms of isoform-specific PI3K signalling. Nat Rev Mol Cell Biol 11:329-341.
34. Vennekens, R., J. Olausson, M. Meissner, W. Bloch, I. Mathar, S. E. Philipp, F. Schmitz, P. Weissgerber, B. Nilius, V. Flockerzi, and M. Freichel. 2007. Increased IgE-dependent mast cell activation and anaphylactic responses in mice lacking the calcium-activated nonselective cation channel TRPM4. Nat Immunol 8:312-320.
35. Vig, M., W. I. DeHaven, G. S. Bird, J. M. Billingsley, H. Wang, P. E. Rao, A. B. Hutchings, M. H. Jouvin, J. W. Putney, and J. P. Kinet. 2008. Defective mast cell effector functions in mice lacking the CRACM1 pore subunit of store-operated calcium release-activated calcium channels. Nat Immunol 9:89-96.
36. Vig, M., C. Peinelt, A. Beck, D. L. Koomoa, D. Rabah, M. Koblan-Huberson, S. Kraft, H. Turner, A. Fleig, R. Penner, and J. P. Kinet. 2006. CRACM1 is a plasma membrane protein essential for store-operated Ca2+ entry. Science 312:1220-1223.
37. Wulff, H., C. Beeton, and K. G. Chandy. 2003. Potassium channels as therapeutic targets for autoimmune disorders. Curr Opin Drug Discov Devel 6:640-647.
38. Zemtsova, I. M., N. Heise, H. Frohlich, S. M. Qadri, Y. Kucherenko, K. M. Boini, D. Pearce, E. Shumilina, and F. Lang. 2010. Blunted IgE-mediated activation of mast cells in mice lacking the serum- and glucocorticoidinducible kinase SGK3. Am J Physiol Cell Physiol 299:C1007-1014.

While certain of the particular embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for screening to identify an inhibitor of phosphatidylinositol-3-kinase C2 beta (PI3KC2β) activity, the method comprising: contacting PI3KC2β or a functional fragment thereof with at least one candidate agent of a plurality of candidate agents and measuring PI3KC2β activity in the presence of the at least one candidate agent, wherein a reduction or inhibition of PI3KC2β activity in the presence of the at least one candidate agent relative to that measured in the absence of a candidate agent or presence of a control agent identifies the at least one candidate agent as the inhibitor of PI3KC2β activity.

2. The method of claim 1, wherein the plurality of candidate agents comprises a library.

3. The method of claim 2, wherein the library is a small molecule or chemical library.

4. The method of claim 1, wherein the contacting is performed in a vessel or in cell culture.

5. The method of claim 4, wherein the vessel is a test tube or a well of a multi-well plate.

6. The method of claim 4, wherein the vessel comprises a solution compatible with PI3KC2β activity.

7. The method of claim 1, further comprising a secondary screen, wherein the inhibitor of PI3KC2β activity identified in a primary screen is assessed with respect to its ability to inhibit other kinases.

8. The method of claim 7, wherein the other kinases are the class I phosphatidylinositol-3-kinase (PI3K) p110α, the epidermal growth factor receptor kinase, ERK map kinase, or Janus Kinase 3.

9. The method of claim 4, wherein the contacting is performed in a vessel, the method further comprising assessing ability of the inhibitor of PI3KC2β activity identified in the vessel to inhibit PI3KC2β activity in cells in cell culture or in a subject.

10. The method of claim 9, wherein the cells in cell culture are primary mast cells generated from bone marrow or peripheral blood in vitro; or a mast cell line.

11. The method of claim 10, wherein the mast cell line is an MC-9 or HC-1 cell line.

12. The method of claim 9, wherein the subject is a mammal afflicted with an IgE-mediated allergic disorder.

13. The method of claim 12, wherein the IgE-mediated allergic disorder is allergic rhinitis, asthma, anaphylaxis, hay fever, eczema, or an immediate type hypersensitivity reaction.

14. A method of treating a subject afflicted with an IgE-mediated allergic disorder, the method comprising administering a therapeutically effective amount of the inhibitor of PI3KC2β activity of claim 1 or a composition thereof to the subject, wherein the administering confers relief from symptoms of the IgE-mediated allergic disorder, thereby treating the subject.

15. The method of claim 14, wherein the IgE-mediated allergic disorder is allergic rhinitis, asthma, anaphylaxis, or an immediate type hypersensitivity reaction.

16. The method of claim 14, wherein the subject is a mammal.

17. The method of claim 16, wherein the mammal is a human.
